(19) United States
(12) Patent Application Publication
(10) Pub. No.: US 2020/0131553 A1
(43) Pub. Date: Apr. 30, 2020

(54) A CHLOROPLAST OR ACCUMULATED LIPID PARTICLE ENRICHED WITH AN OIL-BODY PROTEIN FUSION POLYPEPTIDE AND METHOD FOR PRODUCING THE SAME IN ALGAE

(71) Applicant: Alcantara Research Group Inc., Calgary (CA)

(72) Inventor: Joenel ALCANTARA, Calgary (CA)

(21) Appl. No.: 16/338,874

(22) PCT Filed: Mar. 10, 2017
(86) PCT No.: PCT/CA2017/051172

§ 371 Date: Apr. 2, 2019

Related U.S. Application Data
(60) Provisional application No. 62/403,373, filed on Oct. 3, 2016.

(51) Int. Cl. C12P 21/00; C12N 1/12; C07K 14/405; C07K 1/14

(52) U.S. Cl. CPC C12P 21/00; C12N 1/12; C07K 14/405; C12N 2500/60; C07K 2319/00; C12N 2500/05; C07K 1/14

(57) ABSTRACT

The present invention relates to recombinant protein production in algal cells. In particular, the present invention provides methods for making recombinant polypeptides in association with accumulated lipid particles or chloroplasts. The methods involve producing the recombinant polypeptide as a fusion polypeptide with an oil body protein and the growth of the algal cells under non-homeostatic conditions to form accumulated lipid particles within the algal cells, wherein the algal lipid particles contain the fusion polypeptide.

### FIELD OF THE INVENTION

The present invention pertains to the field of recombinant polypeptide production in algae, and in particular to methods for producing recombinant polypeptide-enriched chloroplasts and accumulated lipid particles.

### BACKGROUND OF THE INVENTION

A wide variety of techniques for the production of recombinant polypeptides in hosts are known in the art. Well-known examples include cell culture based systems, such as microbial cell systems that use bacterial cells, fungal cells, or yeast cells, as well as animal cell systems including mammalian and insect cell culture systems. Other techniques for the production of polypeptides involve the generation of genetically modified plants and animals.

The benefits of using microbial cells to produce recombinant polypeptides include the low cost associated with cultivation of the cells, the potential for substantial product yields, and the general limited toxicity of raw materials. On a larger scale however capital costs may become prohibitively expensive due to factors such as increased material requirements including growth media, scale-up of production facilities, and the expense associated with protein purification, notably in manufacturing operations designed to provide highly purified protein preparations, such as biopharmaceutical proteins.

Historically plants have represented an effective and economical method to produce recombinant polypeptides as they can be grown at a large scale with modest cost inputs. The use of plants has distinct advantages over bacterial systems as bacterial systems are frequently not appropriate for producing many eukaryotic proteins due to differences in protein processing and codon usage. Although foreign proteins have successfully been expressed in plants, the development of systems that offer commercially viable protein yields and are cost effective are still needed. One of the methods which has been explored is the method of production of recombinant polypeptides in association with plant oil-bodies as documented in for example U.S. Pat. No. 5,650,554.

Eukaryotic microalgae, hereinafter “algae” or “algal cells”, are eukaryotic photosynthetic organisms that can readily be grown in a variety of environments, such as large-scale bioreactors, making them attractive candidates for recombinant polypeptide expression.

Techniques to introduce genes capable of expressing recombinant polypeptides in algal cells are well known in the art and research efforts have been made to utilize algae for the purposes of the production of biomolecules as detailed in U.S. Pat. No. 8,951,777; U.S. Pat. No. 9,315,837; U.S. Patent Application No 2011/0030097; U.S. Patent Application No. 2012/0156717; U.S. Pat. No. 6,157,517 and PCT Patent Publication No. WO2012047970.

Algae in principle represent an attractive eukaryotic cellular host system for the synthesis of polypeptides due to the relative ease with which algal cells may be grown, as well as the availability of genetic engineering techniques. In many instances, upon production of the recombinant polypeptide, it is desirable to separate the polypeptide of interest from algal cellular constituents. Known techniques for the isolation of proteins from algal cells include the performance of a wide variety of protein purification techniques, such as chromatographical techniques, including ion exchange chromatography, high performance liquid chromatography, hydrophobic interaction chromatography, and the like. While these techniques are suitable to obtain substantially pure protein preparations on a laboratory scale, they are often inherently impractical to implement on the commercial scale. Moreover, commercial scale protein purification techniques are often the most expensive operational step. Due to the paucity of efficient protein production and extraction techniques known to the art, the commercial manufacture of proteins using algal cells remains substantially economically unviable.

Accordingly, there exists a need for improved techniques for the production of recombinant polypeptides in algae that are readily adaptable to commercial scale operations.

### SUMMARY OF THE INVENTION

An object of the present invention is to provide recombinant polypeptide-enriched chloroplasts or Accumulated Lipid Particles (ALPs) and methods for producing the same in algae. In accordance with an aspect of the invention, there is provided a method of producing a recombinant polypeptide in an algal cell, the method comprising: (a) growing algal cells comprising a recombinant polypeptide under homeostatic conditions to target the recombinant polypeptide to the algal chloroplast; wherein the recombinant polypeptide is a fusion polypeptide comprising an oleosin protein or fragment thereof; and (b) isolating the recombinant protein.

In accordance with another aspect of the invention, there is provided a method of producing algal chloroplasts enriched for recombinant polypeptide, the method comprising: (a) introducing a nucleic acid into algal cells, the nucleic acid comprising as operably linked components (i) a nucleic acid encoding a fusion polypeptide comprising an oleosin protein or fragment thereof to provide targeting to the algal chloroplast and a polypeptide of interest; and (ii) a nucleic acid sequence capable of controlling expression in an algal cell; (b) subjecting the algal cells in a growth medium to homeostatic conditions to target the fusion polypeptide to the algal chloroplast; and (c) optionally isolating the algal chloroplasts.

In accordance with another aspect of the invention, there is provided an algal cell comprising a fusion polypeptide comprising an oleosin or fragment thereof and a protein of interest, wherein the oleosin protein or fragment thereof targets the fusion polypeptide to chloroplasts when the algal cell is subjected to homeostatic conditions and to accumulated lipid particles when the algal cell is subjected to non-homeostatic conditions.

In accordance with another aspect of the invention, there is provided an algal cell comprising nucleic acid comprising as operably linked components (i) a nucleic acid sequence encoding fusion polypeptide comprising an oleosin protein or fragment thereof and a protein of interest, wherein the oleosin protein or fragment thereof targets the fusion polypeptide to chloroplasts when the cell is subjected to homeostatic conditions; and (ii) a nucleic acid sequence capable of controlling expression in an algal cell.

In accordance with another aspect of the invention, there is provided a preparation comprising chloroplasts wherein the chloroplasts comprise a fusion polypeptide comprising an oleosin protein or fragment thereof and a protein of interest, wherein the oleosin protein or fragment thereof targets the fusion polypeptide to chloroplasts when an algal cell is subjected to homeostatic conditions.

In accordance with another aspect of the invention, there is provided a preparation comprising accumulated lipid particles wherein the accumulated lipid particles comprise a fusion polypeptide comprising an oleosin protein or fragment thereof and a protein of interest, wherein the oleosin protein or fragment thereof targets the fusion polypeptide to accumulated lipid particles when the algal cell is subjected to non-homeostatic conditions.

In accordance with another aspect of the invention, there is provided a nucleic acid encoding a fusion polypeptide comprising an oleosin protein or fragment thereof to provide targeting to algal chloroplast under homeostatic conditions and a polypeptide of interest.

In accordance with another aspect of the invention, there is provided a recombinant expression vector comprising a nucleic acid sequence encoding a fusion polypeptide comprising a oleosin protein or fragment thereof to provide targeting to algal chloroplast under homeostatic conditions and to accumulated lipid particle under non-homeostatic conditions and a polypeptide of interest operatively linked to a nucleic acid sequence capable of controlling expression in an algal cell.

In accordance with another aspect of the invention, there is provided a method for producing accumulated lipid particles, the method comprising: (a) introducing a nucleic acid into algal cells, the nucleic acid comprising as operably linked components (i) a nucleic acid encoding a fusion polypeptide comprising an oleosin protein or fragment thereof to provide targeting to the algal chloroplast under homeostatic conditions and accumulated lipid particles under non-homeostatic conditions and a polypeptide of interest; and (ii) a nucleic acid sequence capable of controlling expression in an algal cell; (b) subjecting the algal cell to non-homeostatic conditions to form accumulated lipid particles within the algal cell.

In accordance with another aspect of the invention, there is provided a method of producing a recombinant polypeptide in an algal cell, the method comprising: culturing algal cell comprising fusion polypeptide comprising an oleosin or fragment thereof and a protein of interest, wherein the oleosin protein or fragment thereof targets the fusion polypeptide to chloroplasts when the cell is subjected to homeostatic conditions and accumulated lipid particles when the cell is subjected to non-homeostatic conditions and isolating the recombinant polypeptide.

Other features and advantages of the present disclosure will become apparent from the detailed description. It should be understood, however, that the detailed description, while indicating preferred implementations of the disclosure, are given by way of illustration only, since various changes and modifications within the spirit and scope of the disclosure will become apparent to those of skill in the art of the detailed description.

### BRIEF DESCRIPTION OF THE DRAWINGS

Embodiments of the invention will now be described, by way of example only, by reference to the attached Figures, wherein:

FIG. 1 depicts a map of vector pChlamy_3

FIG. 2 depicts certain cloning steps involved in cloning of a nucleic acid sequence encoding an oleosin-CFP fusion protein where Oleo.FLG.TEV.CFP is inserted into the Kpnl/Xbal of pChlamy_3 vector.

FIG. 3 shows a certain polypeptide sequence of an oleosin-CFP fusion polypeptide (SEQ.ID NO: 22). Highlighted are the oleosin portion and the CFP portion of the fusion polypeptide, as well as a cleavage site and flag tag.

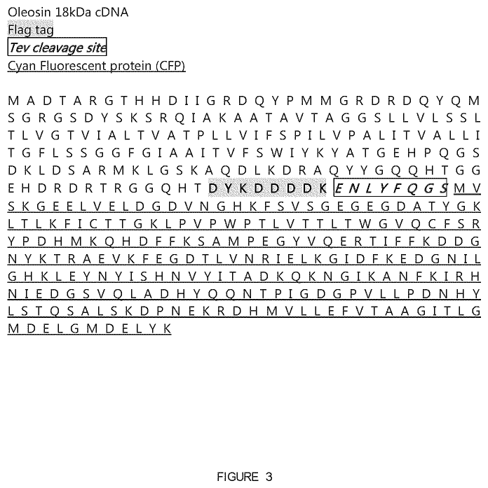

FIG. 4 depicts a map of vector pChlorella.

FIG. 5 shows certain confocal microscopic images and in particular wild type (137c) and transgenic (Oleo1) Chlamydomonas cells containing the Oleo1-CFP construct (under homeostatic (+N) and non-homeostatic (−N) conditions. Viewing conditions were phase contrast (PC), chloroplast autofluorescence (CHL), Cyan Fluorescent Protein (CFP), and Nile Red. In 137c +N cells, there is no CFP signal. There little Nile Red signal, indicating few lipids. In 137c −N, there are several small collections of lipids, indicated by the lighter areas of the image. These areas do not overlap the chloroplast. In transgenic Oleo1 cells under homeostatic conditions, Oleo1-CFP is targeted to the chloroplast. This is indicated by the presence of CFP and chlorophyll autofluorescence in the same areas of the cell. The Nile Red stain for lipids indicated a large amount of lipids present in and associated with the chloroplast. This was indicated by Nile Red signal present in the same area as the CHL panel, and circular structures closely associated with the chloroplast in the Nile Red image. Under stress (−N), Oleo1-CFP is targeted to the ALPs. ALPs are indicated by Nile Red staining for triacylglycerides and CFP. In CFP column images, white arrows indicate areas of CFP where chloroplast autofluorescence is not present.

FIG. 6 shows certain confocal microscopic images and in particular shows results of deleting the hydrophobic domain of Oleo1 to create an OleoNC-CFP strain of Chlamydomonas. Cells were viewed under phase contrast (PC), chloroplast autofluorescence (CHL), Cyan Fluorescent Protein (CFP), and Nile Red under homeostatic (+N) and non-homeostatic (−N) conditions. When the hydrophobic region is deleted and the N and C lobe regions fused to form the OleoNC-CFP construct which is then fused to CFP, the fusion is targeted to the chloroplast under homeostatic conditions. This is demonstrated by the presence of triacylglycerides (indicated in the Nile Red column) and CFP being present in the same areas of the cell as the chloroplast, and being present in parts of the cell other than the chloroplast. Chloroplast is indicated by the fluorescence in the CHL column. Under −N conditions, OleoNC-CFP collects in defined circular areas associated with the chloroplast as shown by white arrows in OleoNC −N Nile Red panel.

FIGS. 7A to 7C shows certain steps in the isolation of ALPs from algal cells. FIG. 7A shows Wild Type 137c TAP, top view; FIG. 7B shows Wild Type 137c TAP-N, top view; and FIG. 7C shows 137c side view; left TAP, right TAP-N.

FIGS. 8A to 8C shows certain steps in the isolation of ALPs from algal cells. FIG. 8A shows Oleo1 TAP, top view, no visible lipid layer; FIG. 8B shows Oleo1 TAP-N, top view, lipid layer covering surface and FIG. 8C shows comparison side view showing significant lipid layer accumulating on top of −N after centrifugation. Arrow indicating lipid layer in TAP-N culture.

FIG. 9 shows certain confocal microscopic images.

FIG. 10 shows depicts wild type and transgenic Chlorella cells containing the Oleo1-CFP construct under homeostatic (CZM1) media) and non-homeostatic (TAP-N media) conditions. Viewing conditions were phase contrast, chloroplast autofluorescence, CFP, and Nile Red. ALPs are indicated by Nile Red staining for triacylglycerides and CFP. In images in CFP column, white arrows indicate areas of CFP where chloroplast autofluorescence is not present. In images in Nile Red column, white arrows indicate areas of Nile Red where chloroplast autofluorescence is not present.

FIG. 11 depicts images of isolated chloroplasts from wild type (137c) and Oleo1 Chlamydomonas cells under phase contrast (PC), chloroplast autofluorescence (CHL), cyan fluorescent protein (CFP), and Nile Red. In column PC, cells are no longer intact in either the 137c or Oleo1 cells. In CHL, there are chloroplasts present in each cell line. In column CFP, there is no signal in 137c while there are areas of CFP expression in the Oleo1 strain. In the CHL+CFP overlay, 137c has autofluorescence but no CFP, while it becomes clear that the CFP expression areas are associated with the chloroplast in Oleo1. Last, there is no Nile Red expression in 137c. Areas of concentrated Nile Red expression in the Oleo1 strain, indicating the presence of lipids, are marked by white arrows. The lipids are present in the same location as the CFP signal, indicating ALPs associated with the chloroplast.

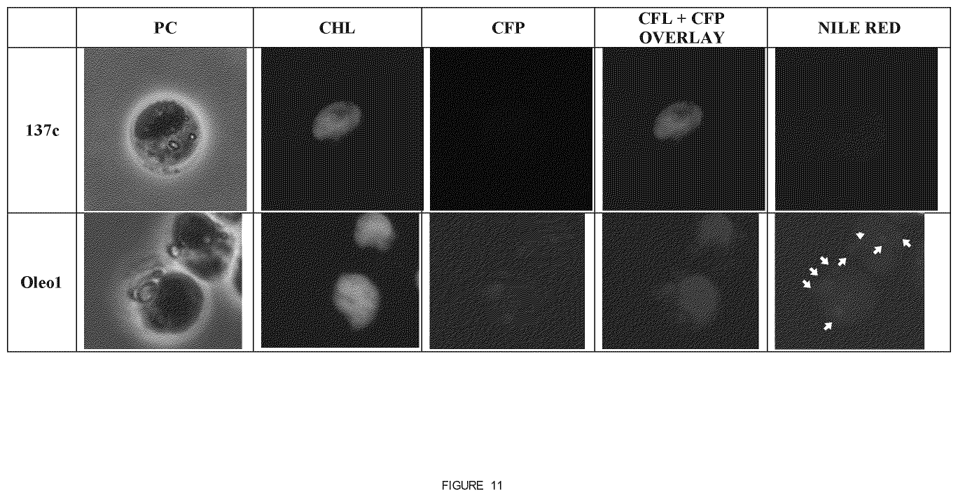

FIG. 12 shows confocal images of the lipids isolated in FIG. 7, and ALPs isolated in FIG. 8. 10 ul of lipids isolated from 137c and 10 ul of ALPs isolated from Oleo1 were applied to a coated slide and imaged as described in Example 3. Images of 137c depict no CFP signal and a very small amount of lipid under Nile Red. Images of Oleo1 depict CFP signal, thus indicating the presence of the Oleosin1-CFP fusion protein. Under Nile Red, large amount of lipids can be seen in the same location as CFP signal, indicating the presence of triacylglycerols and Oleosin1-CFP in the same location.

FIG. 13 shows transgenic Chlamydomonas cells containing the Oleo4-YFP construct under homeostatic (+N) and non-homeostatic (−N) conditions. Viewing conditions were phase contrast (PC), chloroplast autofluorescence (CHL), YFP, and Nile Red.ln transgenic Oleo4-YFP cells under homeostatic conditions, Oleosin4-YFP is targeted to the chloroplast, with small points of high intensity within the chloroplast. This is indicated by the presence of YFP and chlorophyll autofluorescence in the same areas of the cell. Under stress (−N), Oleosin4-YFP is targeted to the ALPs. ALPs are indicated by Nile Red staining for triacylglycerides and YFP. In images in YFP column, white arrows indicate areas of only YFP where chloroplast autofluorescence is not present.

### DETAILED DESCRIPTION OF THE INVENTION

### Definitions

Unless defined otherwise, all technical and scientific terms used herein have the same meaning as commonly understood by one of ordinary skill in the art to which this invention belongs.

The terms “accumulated lipid particle” or “ALP”, as may be used interchangeably herein, refer to subcellular sized approximately spherical particles comprising a triacycl glyceride core encapsulated by oil body proteins or chimeric proteins comprising fragments thereof produced under certain conditions in the transgenic algal cells of the invention.

The term “oil body protein” as used herein are proteins that are naturally associated with plant oil bodies and/or are naturally present on the phospholipid monolayer of plant oil bodies and includes oleosins or functional fragments or derivatives thereof.

The terms “oleosin”, “oleosin protein” and “oleosin polypeptide, as used interchangeably herein, refer to any and all oleosin polypeptides including oleosin 1, oleosin 2, oleosin 3 and oleosin 4 and other oleosin polypeptides. An ordinarily skilled artisan would recognize that oleosin polypeptides may be identified from publicly available databases and include for example those set forth in NCBI Accession: NP_194244, 0A090560, 0A092267 and OAP01777; UniProt

Accession: Q6J1J8; UniProt Accession: Q9XHP2; UniProt Accession: A0A060L102; UniProt Accession: C3S7F7 and UniProt Accession: P13436. Polypeptides also include polypeptides comprising a sequence of amino acid residues which is (i) substantially identical to the amino acid sequences constituting any oleosin polypeptides set forth herein, including those set forth in SEQ.ID NO: 1 to SEQ.ID NO: 6, or (ii) encoded by a nucleic acid sequence capable of hybridizing under at least moderately stringent conditions to the complement of a nucleic acid sequence encoding a oleosin polypeptide set forth herein or a nucleic acid sequence encoding a oleosin polypeptide set forth herein but for the use of synonymous codons.

The term “central domain” as used herein, refers to the “central domain” of any oleosin and includes a membrane-integrating hydrophobic domain. The “central domain” comprises a sequence of amino acid residues which is (i) substantially identical to the amino acid sequences constituting a central domain sequence set forth herein, including the sequences set forth in SEQ.ID NO: 20 and SEQ.ID NO: 24, or (ii) encoded by a nucleic acid sequence capable of hybridizing under at least moderately stringent conditions to the complement of any nucleic acid sequence encoding any central domain set sequence forth herein or to any nucleic acid sequence encoding any central domain polypeptide set forth herein, but for the use of synonymous codons.

The term “proline knot motif”, as used herein, refers to the “proline knot motif” of any oleosin and is found within the hydrophobic domain. The proline knot motif comprises a sequence of amino acid residues which is (i) substantially identical to the amino acid sequences constituting a proline knot motif sequence set forth herein, including those set forth in SEQ.ID NO: 13 to SEQ.ID NO: 18 or (ii) encoded by a nucleic acid sequence capable of hybridizing under at least moderately stringent conditions to the complement of any nucleic acid sequence encoding any proline knot motif set sequence forth herein or any nucleic acid sequence encoding any proline knot motif polypeptide set forth herein, but for the use of synonymous codons.

The herein interchangeably used terms “nucleic acid sequence encoding an oleosin”, “nucleic acid sequence encoding an oleosin protein” and “nucleic acid sequence encoding an oleosin polypeptide”, refer to any and all nucleic acid sequences encoding an oleosin including oleosin 1, oleosin 2, oleosin 3 and oleosin 4 and other oleosin. An ordinarily skilled artisan would recognize that oleosin polynucleotidess may be identified from publicly available databases and include for example those set forth in NCBI Accession number NM_118646.2, NM_123406, NM_124500 and NM 113682; Genbank Accession AY605694.1; Genbank Accession EU999158.1; Genbank Accession KJ415242.2; Genbank Accession EU678264.1; and Genbank Accession M17225.1. Nucleic acids also include those having a sequence set forth in any one of SEQ.ID NO: 7 to SEQ.ID NO: 12. Nucleic acid sequences encoding an oleosin further include any and all nucleic acid sequences which (i) encode polypeptides that are substantially identical to the oleosin amino acid sequences set forth herein; or (ii) hybridize to the complement of any oleosin nucleic acid sequence set forth herein under at least moderately stringent hybridization conditions or which would hybridize thereto under at least moderately stringent conditions but for the use of synonymous codons.

The term “nucleic acid sequence encoding a central domain”, refers to any and all nucleic acid sequences encoding a central domain, including the nucleic acid sequences set forth in SEQ.ID NO: 21 and SEQ.ID NO; 27. Nucleic acid sequences encoding a central domain further include any and all nucleic acid sequences which (i) encode polypeptides that are substantially identical to the central domain sequences set forth herein; or (ii) hybridize to the complement any central domain nucleic acid sequences set forth herein under at least moderately stringent hybridization conditions or which would hybridize thereto under at least moderately stringent conditions but for the use of synonymous codons.

The term “nucleic acid sequence encoding a proline knot motif”, refers to any and all nucleic acid sequences encoding a proline knot motif, including the nucleic acid sequence set forth in SEQ.ID NO: 19. Nucleic acid sequences encoding a proline knot motif further include any and all nucleic acid sequences which (i) encode polypeptides that are substantially identical to the proline knot motif sequences set forth herein; or (ii) hybridize to the complement of any proline knot motif nucleic acid sequences set forth herein under at least moderately stringent hybridization conditions or which would hybridize thereto under at least moderately stringent conditions but for the use of synonymous codons.

By the term “substantially identical” it is meant that two polypeptide or polynucleotide sequences are at least 60% identical, and preferably are at least 85% identical and most preferably at least 95% identical, for example 96%, 97%, 98% or 99% identical.

By “at least moderately stringent hybridization conditions” it is meant that conditions are selected which promote selective hybridization between two complementary nucleic acid molecules in solution. Hybridization may occur to all or a portion of a nucleic acid sequence molecule. The hybridizing portion is typically at least 15 (e.g. 20, 25, 30, 40 or 50) nucleotides in length. Those skilled in the art will recognize that the stability of a nucleic acid duplex, or hybrids, is determined by the Tm, which in sodium containing buffers is a function of the sodium ion concentration and temperature (Tm=81.5° C.-16.6 (Log10 [Na₊])₊0.41(% (G₊C)-60011), or similar equation). Accordingly, the parameters in the wash conditions that determine hybrid stability are sodium ion concentration and temperature. Based on these considerations those skilled in the art will be able to readily select appropriate hybridization conditions. In preferred embodiments, stringent hybridization conditions are selected. By way of example the following conditions may be employed to achieve stringent hybridization: hybridization at 5× sodium chloride/sodium citrate (SSC)/5×Denhardt's solution/1.0% SDS at Tm (based on the above equation)−5° C., followed by a wash of 0.2×SSC/0.1% SDS at 60° C. Moderately stringent hybridization conditions include a washing step in 3×SSC at 42° C. It is understood however that equivalent stringencies may be achieved using alternative buffers, salts and temperatures. Additional guidance regarding hybridization conditions may be found in: Green and Sambrook, Molecular Cloning, a Laboratory Manual, Cold Spring Harbor Laboratory Press, 2012.

The term “homeostatic growth conditions” as used herein, in relation to the cultivation of algal cells, refers to growth conditions under which an algal cell culture is grown under substantially optimal growth conditions. Under homeostatic growth conditions algal cells in a cell culture can temporally exist in different growth phases, including a lag phase; a logarithmic growth phase, also known as exponential growth phase; a stationary phase; or a death phase. During each growth phase, algal cells have a characteristic growth rate corresponding with such growth phase when grown under homeostatic growth conditions. Notably, under homeostatic growth conditions, during logarithmic growth phase, the algal cell population doubles at a constant rate. The rate with which a cell population doubles in size is also known and referred herein as the doubling rate.

The terms “non-homeostatic growth conditions” and “non-homeostatic conditions”, as used herein in relation to the cultivation of algal cells, refers to conditions and growth conditions substantially deviating from homeostatic growth conditions. Under non-homeostatic growth conditions the algal growth rate substantially deviates from the corresponding growth rate under homeostatic growth conditions. When the conditions are altered during the logarithmic phase from homeostatic growth conditions to non-homeostatic conditions the doubling rate decreases to a doubling rate that is lower than the doubling rate during the logarithmic phase under homeostatic growth conditions.

### Overview:

The present invention provides processes and methods of producing recombinant polypeptides in algal cells that are in association with lipid constituents of the cells, notably accumulated lipid particles or chloroplasts depending on growth conditions. By associating the recombinant polypeptides with the lipid constituents or chloroplasts, purification of the recombinant polypeptides may be facilitated since the accumulated lipid particles or chloroplasts and associated recombinant polypeptides can be readily separated from other cellular constituents prior to isolating the recombinant polypeptide. The methods of the invention accordingly eliminate the need for one or more conventional purification steps that are generally required when isolating recombinant polypeptides from algal cells.

The foregoing feature of the methods of the invention can facilitate scale-up and may allow for the economic production of recombinant polypeptides. In particular, the methods are amendable to the production of recombinant polypeptides both at laboratory scale and commercial scale.

As the recombinant polypeptide prior to targeting to the lipid constituents is synthesized in association with the chloroplasts, the recombinant protein is protected from degradation by cytoplasmic enzymes which may afford an advantage with respect to final polypeptide yield.

The method includes subjecting growing algal cells that express an oleosin-fusion polypeptide to specific growth conditions depending on if the recombinant protein is to be targeted to accumulated lipid particles or chloroplasts.

In one embodiment, under homeostatic conditions the recombinant polypeptide is targeted to chloroplasts and under non-homeostatic conditions the recombinant polypeptide is targeted to accumulated lipid particles. The fusion polypeptide includes an oleosin protein or fragment thereof that targets the fusion polypeptide to the lipid constituents and particularly accumulated lipid particles following stress or non-homeostatic conditions. In some embodiments, the fusion polypeptide includes a Oleo1, Oleo2, Oleo3, or Oleo4 protein or a targeting fragment thereof.

In some embodiments, more than one oleosin protein or fragment thereof is used to target the protein of interest. In some embodiments, the algal cells may include more than one fusion protein each optionally have a different targeting oleosin protein or fragment.

In some embodiments, the oleosin protein or targeting fragment comprises fragments of more than one type of oleosin protein and/or has been modified.

The recombinant polypeptide enriched accumulated lipid particles or chloroplasts may be isolated from the algae by various techniques known in the art. Optionally, the recombinant polypeptides are isolated from the accumulated lipid particles or chloroplasts. Techniques for isolating the polypeptides from the accumulated lipid particles or chloroplasts are also known in the art.

Alternatively, the recombinant polypeptide enriched accumulated lipid particles or chloroplasts are isolated for use in nutraceutical, pharmaceutical or other applications known in the art.

In some embodiments, the recombinant protein enriched accumulated lipid particles or chloroplast may be orally ingested. Such encapsulation will protect the recombinant polypeptide from, for example, digestive processes that may degrade the polypeptide, preventing it from performing its biological function.

A worker skilled in the art would readily appreciate that the methods of the invention can be used with any or all eukaryotic, microalgal cells or algae including, without limitation any algae classified as green algae (Chlorophyceae), diatoms (Bacillariophyceae), yellow-green algae (Xanthophyceae), golden algae (Chrysophyceae), red algae (Rhodophyceae), brown algae (Phaeophyceae), dinoflagellates (Dinophyceae) or pico-plankton (Prasinophyceae and Eustigmatophyceae). Examples of algal cells further include any algal species belonging to the genus, Clamydomonas, for example Chlamydomonas reinhardtii, and any algal species belonging to the genus Chlorella.

In one embodiment, the algal cell is a green algae (Chlorophyceae).

In one embodiment, the algal cell is a diatom (Bacillariophyceae).

In one embodiment, the algal cell is a yellow-green algae (Xanthophyceae).

In one embodiment, the algal cell is a golden algae (Chrysophyceae).

In one embodiment, the algal cell is a red algae (Rhodophyceae).

In one embodiment, the algal cell is a brown algae (Phaeophyceae).

In one embodiment, the algal cell is a dinoflagellates (Dinophyceae).

In one embodiment, the algal cell is a pico-plankton (Prasinophyceae and Eustigmatophyceae).

In one embodiment, the algal cell is an algal species belonging to the genus Clamydomonas, including but not limited to Chlamydomonas reinhardtii.

In one embodiment, the algal cell is an algal species belonging to the genus Chlorella.

In some embodiments, mixtures of eukaryotic algal species can be used, including but not limited to species belonging to any of the aforementioned.

In some embodiments, the algal cells are transgenic algae cells that are further modified. In some embodiments, the transgenic algae cells include a transgene, vector or like that is controlled by the recombinant protein of the invention.

### Recombinant Polypeptides and Polynucleotides

The present invention provides for recombinant polypeptides that can be targeted to accumulated lipid particles or chloroplasts in response to growth conditions.

Targeting to accumulated lipid particles or chloroplasts in response to growth conditions is a result of fusion of interest to an oleosin protein. In certain embodiments, the targeting polypeptide is oleosin, a derivative or fragment thereof. Optionally, the oleosin, derivative or fragment thereof is Oleo1, Oleo2, Oleo3, Oleo4 or other oleosin known in the art or derivatives or fragments or combinations thereof. Example oleosin polypetide sequences that may be used include the following: SEQ.ID NO: 1-SEQ.ID NO: 6 and SEQ. ID NO: 34-36.

In some embodiments, more than one oleosin protein or fragment thereof is used to target the protein of interest. In some embodiments, the algal cells may include more than one fusion protein each optionally have a different targeting oleosin protein or fragment.

In some embodiments, the oleosin protein or targeting fragment comprises fragments of more than one type of oleosin protein and/or has been modified.

In some embodiments, the targeting polypeptide includes substantially the full length oleosin. In other embodiments, the targeting polypeptide excludes part of or all of the hydrophobic domain, optionally the targeting polypeptide is Oleo1 excluding the hydrophobic domain.

The present invention provides nucleic acid sequence encoding a fusion polypeptide comprising a portion of an oil body protein capable targeting of the fusion polypeptide to the algal chloroplast linked to a polypeptide of interest to lipid particles or chloroplasts in response to growth conditions. The nucleic acid may further include nucleic acid sequences capable of controlling expression in an algal cell.

In one embodiment, the nucleic acid encodes a sufficient portion of an oil body protein to provide targeting of the fusion polypeptide is an intact oil body protein, including an intact oleosin. Example nucleic acid sequences encoding oleosins that may be used include the following: SEQ.ID NO: 7-SEQ.ID NO: 12 and SEQ. ID NO: 37-39. Further oil body proteins that may be used in accordance herewith are oleosins obtainable or obtained from an oil seed plant including, without limitation, thale cress (Arabidopsis thalania), soybean (Glycine max), rapeseed (Brassica spp.), sunflower (Heliantus annuus), safflower (Carthamus tinctorius, mustard (Brassica spp and Sinapis alba) and maize (Zea mays). In some embodiments, the nucleic acid sequences have been codon optimized for the specific algae.

In other embodiments, the nucleic acid encoding a sufficient portion of an oil body protein to provide targeting of the fusion polypeptide is a portion of an oil body protein, including a portion of an oleosin. In some embodiments, the portion of an oil body protein providing targeting comprises or consists of the central domain of an oleosin polypeptide. Example nucleic acid sequences encoding the central domain of an oleosin include SEQ.ID NO: 21 and SEQ.ID NO: 27.

In some embodiments, the portion of an oil body protein providing targeting of the fusion polypeptide comprises or consists of a proline knot motif. SEQ.ID NO: 13 to SEQ.ID NO: 19 (oleosin).

In some embodiments, the portion of the oil body protein providing targeting comprises or consists of the central domain or a proline knot motif of an oleosin and the N-terminal domain of an oleosin. Example nucleic acid sequences encoding an N-terminal domain of an oleosin include SEQ.ID NO: 26. In some embodiments, the portion of the oil body protein providing targeting comprises or consists of the C-terminal domain of an oleosin. Example nucleic acid sequences encoding a C-terminal domain of an oleosin include SEQ.ID NO: 28.

In some embodiments, the portion of the oil body protein providing targeting comprises the N-terminal domain of an oleosin and C-terminal domain of oleosin. Example nucleic acid sequences encoding an N-terminal domain of an oleosin include SEQ.ID NO: 26. Example nucleic acid sequences encoding a C-terminal domain of an oleosin include SEQ.ID NO: 28.

The nucleic acid encoding a recombinant polypeptide may be any nucleic acid encoding a recombinant polypeptide, including any intact polypeptide of any length, varying from several amino acids in length to hundreds of amino acids in length, or any fragment or variant form of an intact recombinant polypeptide. In addition, in some embodiments, the nucleic acid encoding the polypeptide of interest may encode multiple polypeptides of interest, for example, a first and a second recombinant polypeptide, which may be linked to one another.

The recombinant polypeptide of interest may be any recombinant polypeptide including, without limitation insulin, hirudin, an interferon, a cytokine, an immunoglobulin, an antigenic polypeptide, a hemostatic factor, such as Willebrand Factor, a peptide hormone, such as angiotensin, β-glucuronidase (GUS), factor H binding protein, gam56, VP2, cellulase, xylanase, a protease, chymosin and chitinase.

As will readily be appreciated by those of skill in the art, depending on the nucleic acid sequence encoding the recombinant polypeptide, a wide variety of polypeptides may be selected and obtained, and the utility of the selected recombinant polypeptide may vary widely. Nucleic acid sequences encoding recombinant polypeptides may be identified and retrieved from databases such as GenBank (http://www.ncbi.nlm.nih.gov/genbank/) or nucleic acid sequences may be determined by methods such as gene cloning, probing and DNA sequencing. In accordance herewith, the nucleic acid sequence encoding the recombinant polypeptide may be selected in accordance with any and all applications for which the selected polypeptide is deemed useful. The actual nucleic acid sequence of the polypeptide of interest in accordance with the present disclosure is not limited, and may be selected as desired. In accordance herewith such recombinant polypeptides may be any polypeptides for use in pharmaceutical and biopharmaceutical or veterinary applications, any polypeptides for use in food, feed, nutritional and nutraceutical applications, any polypeptides for use in cosmetic and personal care applications, any polypeptides for use in agricultural applications, any polypeptides for use in industrial or domestic applications, any polypeptides that may be beneficial for algal growth, for example enzymes providing herbicidal or antibiotic resistance, and recombinant polypeptides for any other uses one desires to produce in accordance in accordance with the present disclosure.

In some embodiments, the 3′ end of the nucleic acid sequence encoding the sufficient portion of a polypeptide to provide targeting to an oil body is linked to the 5′ end of the nucleic acid sequence encoding the polypeptide of interest.

In some embodiments, the 5′ end nucleic acid sequence encoding the sufficient portion of a polypeptide to provide targeting to an oil body is linked to the 3′ end of the nucleic acid sequence encoding the polypeptide of interest.

In some embodiments, both the 5′ end and the 3′ end of the nucleic acid sequence encoding a sufficient portion of a polypeptide to provide targeting to an oil body are linked to the 3′ end of a nucleic acid sequence encoding the polypeptide of interest and to the 5′ end of a nucleic acid sequence encoding a polypeptide of interest, respectively. In this embodiment, the two recombinant polypeptides of interest may be identical or different.

In some embodiments, the 3′ end of a first nucleic acid sequence encoding a sufficient portion of a polypeptide to provide targeting of a fusion polypeptide is linked to the 5′ end of a nucleic acid sequence encoding a polypeptide of interest and the 3′ end of the same nucleic acid sequence encoding a polypeptide of interest is linked to the 5′ end of a second nucleic acid sequence encoding a sufficient portion of a polypeptide to provide targeting of to a fusion polypeptide.

In some embodiments, the nucleic acid sequence encoding a sufficient portion of an oil body protein to provide targeting to an oil body is separated from the nucleic acid sequence encoding by a cleavable peptide linker sequence. In some embodiments, the cleavable peptide linker sequence is enzymatically cleavable, for example a linker sequence cleavable by enzymes such as thrombin, Factor Xa collagenase, or chymosin. An example of a linker sequence that may be used includes: SEQ.ID NO: 29 (encoded by SEQ.ID NO:30). In other embodiments, the cleavable peptide linker sequence is chemically cleavable, for example cyanogen bromide. In further embodiments, the nucleic acid sequence further comprises a nucleic acid sequence that permits autocatalytic cleavage, for example, a nucleic acid sequence encoding a chymosin or an intein (SEQ.ID NO: 31 (encoded by SEQ.ID NO: 32).

Nucleic acid sequences encoding fusion polypeptides can be prepared using any technique useful for the preparations of such nucleic acid sequences and generally involves obtaining a nucleic acid sequence encoding a sufficient portion of an oil body protein to target the fusion polypeptide, and a nucleic acid sequence encoding recombinant polypeptide of interest, for example by synthesizing these nucleic acid sequences, or isolating them from a natural source, and then linking the two nucleic acid sequences, using for example nucleic acid cloning vectors, such as the pUC and pET series of cloning vectors, microbial cloning host cells, such as Escherichia coli, and techniques such as restriction enzyme digestion, ligation, gel-electrophoresis, polymerase chain reactions (PCR), nucleic acid sequencing, and the like, which are generally known to those of skill in the art. Additional guidance regarding the preparation of nucleic acid sequences encoding fusion polypeptides including the use and cultivation of E. coli as a microbial cloning host may be found in: Green and Sambrook, Molecular Cloning, a Laboratory Manual, Cold Spring Harbor Laboratory Press, 2012 and Esposito et al., 2009 Methods Mol. Biol. 498: 31-54.

In accordance with one aspect hereof, the nucleic acid sequence encoding a fusion polypeptide is linked to a nucleic acid sequence capable of controlling expression in an algal cell. Accordingly, the present disclosure also provides, in one embodiment, a nucleic acid sequence encoding a fusion polypeptide comprising a sufficient portion of an oil body protein to provide growth condition dependent targeting of the fusion polypeptide to accumulated lipid particles or chloroplast linked to a recombinant polypeptide; and a nucleic acid sequence capable of controlling expression in an algal cell.

Nucleic acid sequences capable of controlling expression in algal cells that may be used herein include any transcriptional promoter capable of controlling expression of polypeptides in algal cells. Generally, promoters obtained from algal cells are used, including promoters associated with lipid production in algal cells. Promoters may be constitutive or inducible promoters, for example an oxygen inducible promoter.

Examples of transcriptional promoters that may be used in accordance herewith include SEQ.ID NO: 33. Further nucleic acid sequence elements capable of controlling expression in an algal cell include transcriptional terminators, enhancers and the like, all of which may be included in the chimeric nucleic acid sequences of the present disclosure.

In accordance with one aspect of the present disclosure, the nucleic acid comprising a nucleic acid sequence capable of controlling expression in algal cell linked to a nucleic acid sequence encoding a fusion polypeptide comprising a sufficient portion of oleosin to provide targeting of the fusion polypeptide to an accumulated lipid particle linked to a recombinant polypeptide, can be integrated into a recombinant expression vector which ensures good expression in the algal cell.

The term “suitable for expression in an algal cell”, as used herein, means that the recombinant expression vector comprises the nucleic acid sequence of the present disclosure linked to genetic elements required to achieve expression in an algal cell. Genetic elements that may be included in the expression vector in this regard include a transcriptional termination region, one or more nucleic acid sequences encoding marker genes, one or more origins of replication and the like. The genetic elements are operably linked, typically as well be known to those of skill in the art, by linking e.g. a promoter in the 5′ to 3′ direction of transcription to a coding sequence. In preferred embodiments, the expression vector may further comprise genetic elements required for the integration of the vector or a portion thereof in the algal cell's genome.

Pursuant to the present disclosure, the expression vector can further contain a marker gene. Marker genes that may be used in accordance with the present disclosure include all genes that allow the distinction of transformed algal cells from non-transformed cells, including all selectable and screenable marker genes. A marker gene may be an antibiotic resistance marker against, for example, kanamycin, spectinomycin, hygromycin or zeocin. Further markers include herbicide resistance markers such as norflurazon, or metabolic markers that necessitate addition of substances to the media for growth such as in the case of arginine auxotrophy mutations. Screenable markers that may be employed to identify transformants through visual inspection include, β-galactosidase, β-glucuronidase (GUS) (U.S. Pat. Nos. 5,268,463 and 5,599,670), green fluorescent protein (GFP) (Niedz et al., 1995, Plant Cell Rep 14:403-406), and other fluorescent proteins.

To assemble the expression vector, an intermediary cloning host can be used. One intermediary cloning host cell that particularly conveniently can be used is E. coli using various techniques that are generally known to those of skill in the art including hereinbefore mentioned techniques for cloning and cultivation and general guidance that can for example be found in Sambrook et al., Molecular Cloning, a Laboratory Manual, Cold Spring Harbor Laboratory Press, 2001, Third Ed.

To introduce the chimeric nucleic acid sequence in algal cells, algal cells can be transformed using any technique known to the art, including, but not limited to, biolistic bombardment, glass beads, autolysin assisted transformation, electroporation, silicon carbide whiskers (Dunahay, T. G. (1993). BioTechniques 15, 452-460. Dunahay , T. G. , Adler, S. A., and Jarvik , J. W. (1997). Methods Mol. Biol. 62, 503-509), Agrobacterium-mediated gene transfer, and sonication or ultrasonication. The selected transformation technique can be varied depending on the algal species selected. In embodiments in which the selected algal cells lack a cell wall, glass bead transformation method is preferred. In the performance of this method, in general, glass beads containing the chimeric nucleic acid sequence, for example a linearized chimeric nucleic acid sequence, are placed in a reaction tube with an algae cell suspension and the mixture is vigorously vortexed to effect uptake of the chimeric nucleic acid sequence by the algal cells (Kindle, K. L., (1990). Proc. Natl. Acad. Sci (USA) 87, 1228 -1232). In embodiments in which the algal cells have cell walls, autolysin assisted transformation is a preferred methodology. In general, autolysin assisted transformation methodology, involves the incubation of algal cells with autolysin, an enzyme which naturally digests the cell wall during cellular mating and renders the algal cells susceptible to the receipt of nucleic acid material (Nelson et al., Mol. Cell Biol. 14: 4011-4019). In the performance of electroporation based techniques, an electric field is applied to the algal host cells to induce membrane permeability to effect uptake by the algal cells of the nucleic acid. Electroporation is a particularly preferred methodology since many algal species are readily susceptible to uptake of nucleic acid material upon electroporation (Brown et al., Mol. Cell Biol. (1991) 11 (4) 2382-2332 (PMC359944). In certain embodiments biolistic bombardment is used. In the performance of biolistic bombardment based techniques, in general, a particle delivery system is used to introduce the chimeric nucleic acid sequence into algae cells (Randolph-Anderson et al., BioRad Technical

Bulletin no 2015 [http://www.bio-medicine.org/biology-technology/Sub-Micron-Gold-Particles-Are-Superior-to-Larger-Particles-for-Efficient-Biolistic-Transformation-of-Organelles-and-Some-Cells-1201-1/]. A further methodology that can be used to obtain transformed algal cells is Agrobacterium tumefaciens mediated transformation, which in general involves the infection of algal cells with Agrobacterium cells transformed to contain the chimeric nucleic acid sequence and upon infection transfer of the chimeric nucleic acid sequence to algal cells Kumar, S. V. et al. (2004). Genetic transformation of the green alga Chlamydomonas reinhardtii by Agrobacterium tumefaciens. Plant Sci. 166, 731-738. Yet one further methodology that in certain embodiments can be used is the use of ultrasound mediated delivery of the chimeric nucleic acid sequence into algae as is for example described in Unites States Patent Application no. US2015/0125960.

In some embodiments, upon introduction of the chimeric nucleic acid, the chimeric nucleic acid may be incorporated in the genome of the algal cell, generally resulted in inheritable expression. To facilitate integration in the genome of the algal cell, the chimeric nucleic acid may comprise one or more nucleic acid sequences that facilitate integration of the chimeric nucleic acid sequence in the algal genome.

In some embodiments, upon introduction of the chimeric nucleic acid, the chimeric nucleic acid may be maintained as a chimeric nucleic acid outside of the genome of the algal cell, generally resulting in transient expression.

### Growth Conditions

Under homeostatic growth conditions, the fusion protein is targeted to the chloroplasts. To target the fusion protein to the accumulated lipid particles from chloroplasts, the algal cells comprising the fusion proteins are subjected to stress or non-homeostatic growth condition.

In accordance with certain embodiments, the algal cells are grown in a growth medium under homeostatic growth conditions to target the recombinant polypeptide to the chloroplast within the algal cells.

In accordance with certain embodiments, the algal cells are grown in a growth medium under non-homeostatic growth conditions to form accumulated lipid particles within the algal cell, wherein the accumulated lipid particles comprise the fusion polypeptide.

In some embodiments, the algal cells are initially grown under homeostatic growth conditions wherein substantially no accumulated lipid particles are formed, and subsequently grown under non-homeostatic growth conditions.

Growth of algal cells under homeostatic conditions can be performed using any growth media suitable for the growth of algal cells, comprising non-limiting amounts of nutrients, including nutrients providing a carbon source, a nitrogen source, and a phosphorus source, as well as trace elements such as aluminum, cobalt, iron, magnesium, manganese, nickel, selenium zinc, and the like, and growing algal cells under optimal growth conditions. Conditions to achieve homeostatic growth for algal cells vary depending on the selected algal species, however such conditions typically include temperatures ranging, from 20° C. to 30° C., light intensities varying from 25-150 μE m⁻² s⁻¹ and a pH that is maintained in a range from 6.8 to 7.8.

Homeostatic growth conditions also include conditions appropriate for batch cultivation of algal cells, as well as conditions for continuous algal cell cultivation. In some embodiments, liquid culture media are used to grow the algal cells. In alternate embodiments, solid media for algal growth may also be used as a substrate for algal growth (The Chlamydomonas Sourcebook (Second Edition) Edited by:Elizabeth H. Harris, Ph.D., David B. Stern, Ph.D., and George B. Witman, Ph.D. ISBN: 978-0-12-370873-1) Further guidance to prepare suitable media for the homeostatic growth of algae, as well as guidance to suitable culturing conditions for algae are further described in Appl Microbiol Biotechnol. 2014 Jun;98 (11):5069-79. doi: 10.1007/s00253-014-5593-y. Epub 2014 Mar 4; Handbook of Microalgal Culture: Applied Phycology and Biotechnology By Amos Richmond, Qiang Hu ISBN 140517249; and in Algal Culturing Techniques Robert Arthur Anderson 2005 ISBN 0120884267. The concentration of a nutrient and/or a growth condition may be optimized or adjusted, for example by preparing a plurality of growth media, each including a different concentration of a nutrient, growing algal cells in each of the growth media, and evaluating algal growth, example, by evaluating cell density as a function of time. Then, a growth medium or growth condition can be selected that provides the most desirable effect.

In accordance with one aspect hereof, the algal cells are subjected to non-homeostatic conditions. Non-homeostatic conditions may be selected for the specific algae strain and/or selected based on the growth of the non-transgenic parent strain under those conditions. By “subjecting to non-homeostatic conditions”, it is meant that the conditions under which the algal cells are grown are gradually or abruptly modulated, or established in such a manner that algal cell growth rates substantially deviate from growth rates under homeostatic growth conditions. Thus, for example, the algal cell growth rate during log phase growth under homeostatic growth conditions deviates substantially from the algal cell growth rate during log phase growth under non-homeostatic conditions, and the algal cell growth rate during stationary phase growth under homeostatic growth conditions deviates substantially from the algal cell growth rate under non-homeostatic conditions. Substantial deviations include deviations wherein the growth rate under a non-homeostatic condition is less than about 0.8 or 0.8, about 0.7 or 0.7, about 0.6 or 0.6, about 0.5 or 0.5, about 0.4 or 0.4, about 0.3 or 0.3, about 0.2 or 0.2, or about 0.1 or 0.1 times the growth rate under a corresponding homeostatic growth condition.

In some embodiments, the algal cells immediately following introduction of the nucleic acid sequence within the algal cells are grown under non-homeostatic conditions. In some embodiments, the cells are grown or maintained in lag phase and not permitted to enter logarithmic phase.

In some embodiments, the algal cells, for example immediately following the introduction of the nucleic acid sequence, are initially grown under homeostatic conditions, and are then subjected to non-homeostatic conditions to grow or maintain the algal cells under non-homeostatic conditions.

In one embodiment, the algal cells are grown to logarithmic phase, and while in logarithmic phase the cells are subjected to non-homeostatic growth conditions to grow or maintain the algal cells under non-homeostatic conditions. Thus in this embodiment, the doubling rate decreases from a logarithmic doubling rate to a doubling rate that is substantially lower than the doubling rate under logarithmic homeostatic conditions, for example, the doubling rate under non-homeostatic conditions is less than about 0.8 or 0.8, about 0.7 or 0.7, about 0.6 or 0.6, about 0.5 or 0.5, about 0.4 or 0.4, about 0.3 or 0.3 , about 0.2 or 0.2, or about 0.1 or 0.1 times the doubling rate under homeostatic growth conditions during logarithmic phase. In some embodiments, the doubling rate, upon subjecting the cells to non-homeostatic conditions may alter from a constant doubling rate to a declining doubling rate.

In some embodiments, upon subjecting the cells to non-homeostatic conditions, the cells may enter a different growth phase, for example the cells may enter stationary growth phase from logarithmic phase.

In one embodiment, non-homeostatic growth conditions are conditions in which one or more nutrients are present in the algal cell growth medium in quantities that are insufficient for homeostatic algal cell growth.

In one embodiment, non-homeostatic growth conditions are conditions in which nitrogen is present in the algal cell growth medium in quantities that are insufficient for homeostatic algal cell growth. In some embodiments, the quantities of nitrogen present in the medium to for non-homeostatic growth ranges from about 0 mole/liter to about 0.02 mole/liter.

In one embodiment, non-homeostatic growth conditions are conditions in which phosphorus is present in the algal cell growth medium in quantities that are insufficient for homeostatic algal cell growth. In some embodiments, the quantities of phosphorus present in the medium for non-homeostatic growth ranges from about 0 to 0.8 mM.

In another embodiment, an exogenous stress factor, for example a physical, chemical or biological stress factor, is applied to an algal cell culture comprising a chimeric nucleic acid sequence of the present disclosure to effect non-homeostatic conditions.

In one embodiment, the exogenous stress factor applied is an adjustment of the pH of an algal cell culture to obtain a growth medium having non-homeostatic pH, and growing the cells at a non-homeostatic pH. In some embodiments, the pH is adjusted in such a manner that the pH of the algal culture ranges between about pH 5.0 to 6.5.

In one embodiment, the exogenous stress factor applied is an adjustment of the salinity of an algal cell culture to obtain a growth medium having a non-homeostatic salinity, and growing the cells under non-homeostatic salinity. In some embodiments, the salinity is adjusted in such a manner that the concentration of sodium and chloride ions of the algal culture ranges between about 20 to about 200 mM.

In one embodiment, the exogenous stress factor applied is an adjustment of the light intensity to which an algal cell culture is exposed to obtain a growth condition having a non-homeostatic light intensity, and growing the cells under non-homeostatic light intensity. In some embodiments, the light intensity is adjusted in such a manner that the light intensity to which the algal culture is exposed ranges between about 150-1000 μE m⁻² s⁻¹ .

Non-homeostatic growth conditions may be detected and measured by comparing growth of algal cells under homeostatic conditions with growth of algal cells under non-homeostatic conditions. Thus, for example, the cell density of an algal cell culture may be determined, for example, by determining the optical density, or a cell counter such as a Coulter counter or flow cytometer, or manually counting cells using a hemocytometer, and the densities of algal cell cultures grown under homeostatic and non-homeostatic growth conditions may be compared. By measuring the cell density at different time points the growth rate and doubling rate of an algal cell culture, whether grown under homeostatic or non-homeostatic conditions, may be determined. Further guidance with respect to measuring algal cell growth may be found in The Chlamydomonas Sourcebook (Second Edition) Edited by: Elizabeth H. Harris, Ph.D., David B. Stern, Ph.D., and George B. Witman, Ph.D. (ISBN: 978-0-12-370873-1).

In accordance with one aspect, upon growth under non-homeostatic conditions, the algal cells produce accumulated lipid particles comprising a fusion polypeptide comprising the recombinant polypeptide.

In accordance with one embodiment, synthesis of the accumulated lipid particles produced by the algal cells when the cells are grown under non-homeostatic conditions, originate at the algal chloroplasts. Production of lipids, including in association with chloroplasts and accumulated lipid particles may be evaluated by staining algal cells with a lipophilic stain, such as Nile Red.

In accordance with one embodiment, the fusion polypeptide is upon introduction of the chimeric nucleic acid sequence in the algal cell first produced by the algal cell in association with the algal chloroplasts and thereafter, and upon subjecting the algal cells to non-homeostatic conditions, the fusion polypeptide is targeted to the accumulated lipid particles.

In accordance with one embodiment, hereof the fusion polypeptide is produced in association with the algal chloroplasts and the accumulated lipid particles and the fusion polypeptide is protected from exposure to the cytoplasm, and from degradation by cytoplasmic enzymes.

In some embodiments, targeting of the fusion polypeptide may be evaluated, for example using techniques such as electron microscopy, and confocal fluorescent microscopy in conjunction with fluorescent antibodies having a specificity for the recombinant polypeptide of interest.

In different embodiments, the algal cells may be subject to different non-homeostatic conditions, as herein before described, e.g. in the the presence of quantities of nutrients, such as nitrogen, or phosphate in quantities that are insufficient for homeostatic growth, or by subjecting the cells to an exogonous stress factor e.g. non-homeostatic pH conditions, non-homeostating light conditions or non-homeostatic salinity etc.

In some embodiments, the recombinant algal cells are grown at temperatures over 22° C. and with or without CO₂ over 0.5% to facilitate clumping.

In some embodiments, the clumping of recombinant algal cells may be facilitated by the addition of chemical additives to the media as is known in the art.

### Harvesting

Recombinant protein can be isolated from either chloroplasts or from accumulated lipid particles depending on growth condition dependent targeting. In some embodiments, algal cells are grown under homeostatic conditions such that recombinant protein is targeted to chloroplasts. In some embodiments, algal cells are grown under non-homeostatic conditions prior harvesting such that recombinant protein is targeted to accumulated lipid particles.

Algal cells may be harvested by a variety of techniques known in the art including centrifugation and filtration. Optionally, harvesting includes a flocculation step where clumping of algal cells is promoted by growth conditions and/or additives and/or other methods known in the art.

In accordance with some embodiments where the harvesting of algal cells includes a flocculation step, the algal clumps are isolated.

In some embodiments after the algal cells are harvested, chloroplasts or accumulated lipid particles are isolated.

In accordance with one embodiment, chloroplasts comprising recombinant protein are isolated from the algal cells. Methodologies for the isolation of chloroplasts from algae will generally be known to those of skill in the art, and include but are not limited to the methodologies described in Mason, et al. (2006). Nat. Protoc. 1, 2227-2230.

In accordance with one embodiment, the accumulated lipid particles may be isolated. This may generally be accomplished by harvesting the algal cells by separating the algal cells from the growth medium. Thereafter the algal cells can be disrupted, using for example chemical techniques, such as enzymatic digestion, and/or physical techniques such as homogenization, sonication, and/or glass or ceramic beads to obtain a suspension comprising disrupted algal cells. The foregoing techniques are generally able to disrupt algal cell walls and membranes however they are relatively gentle in order to avoid compromising the integrity of the accumulated lipid particles. The accumulated lipid particles subsequently can readily be separated from other aqueous cell constituents.

In some embodiments, separation of the accumulated lipid particles is by density based separation techniques, for example centrifugation. Upon centrifugation of the suspension comprising disrupted algal cells a two-phase suspension comprising in a first phase the accumulated lipid particles and in a second phase the aqueous cellular constituents can be obtained from which the first phase comprising the accumulated lipid particles can be readily separated and removed. In some embodiments, accumulated lipid particles are isolated from cooled algal cells, optionally the algal cells cooled to 4° C.

The foregoing procedures may readily be conducted on a laboratory scale or on a larger commercial scale.

In some embodiments, the recombinant protein is isolated from the chloroplasts or accumulated lipid particles.

In accordance with one embodiment, the fusion polypeptide may include a cleavable linker sequence and upon isolation of the chloroplasts the recombinant polypeptide may be separated from the chloroplasts and the oil body protein, or portion thereof, as the case may be, and a substantially pure recombinant polypeptide may be obtained, using any protein purification methodology, including without limitation, those hereinbefore described.

The recombinant polypeptide may be recovered or isolated by a variety of different protein purification techniques including, e.g. metal-chelate chromatography, ion-exchange chromatography, size exclusion chromatography, affinity chromatography, hydrophobic interaction chromatography, reverse phase chromatography, gel filtration, etc. Further general guidance with respect to protein purification may for example be found in: Protein Purification: Principles, High Resolution Methods, and Applications, Janson, 2013, vol. 54. Wiley.

### EXAMPLES

Hereinafter are provided examples of specific implementations for performing the methods of the present disclosure, as well as implementations representing the compositions of the present disclosure. The examples are provided for illustrative purposes only, and are not intended to limit the scope of the present disclosure in any way.

### Example 1

### Vector Construction

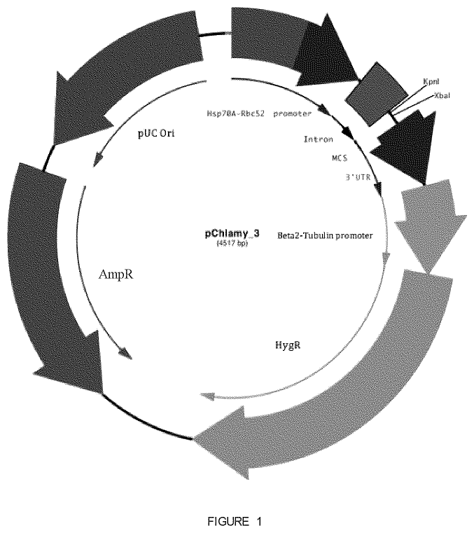

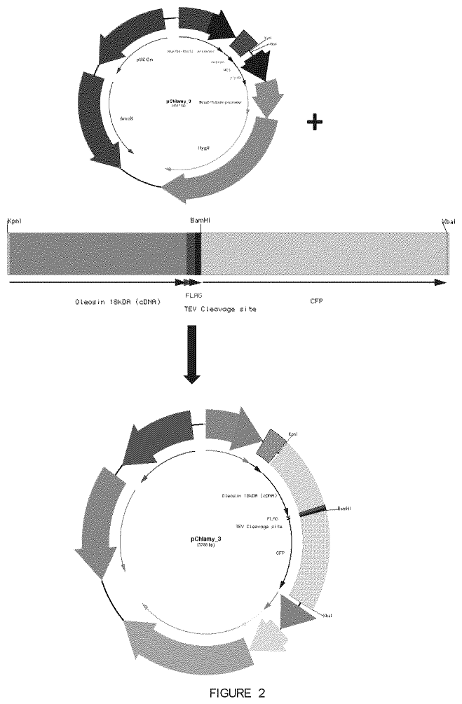

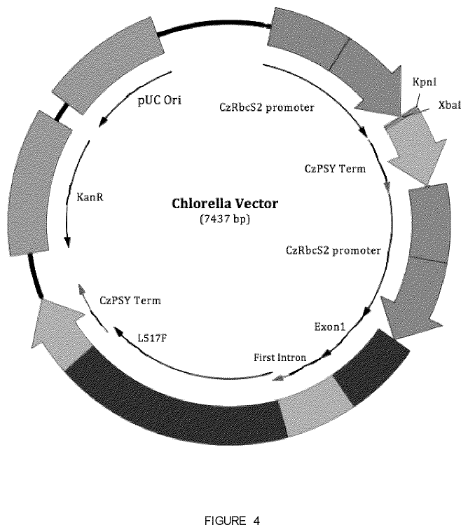

This example describes the construction of one Chlamydomonas vector (pChlamy_3, shown in FIG. 1, and FIG. 2) and one Chlorella vector (FIG. 4).

pChlamy_3 plasmid was obtained from Invitrogen. To create the Chlorella vector, The C. reinhardtii PDS gene (CrPDS) containing the modified amino acids (L131V,L505F) was synthesized by GenScript and cloned into the pUC57-Kan vector. All standard recombinant DNA techniques (DNA digestion by restriction endonucleases, DNA ligation, plasmid isolation, and preparation of media and buffers) were performed as previously described (Sambrook, Fritsch, & Maniatis, 1989). The restriction endonucleases, BamHI, Kpnl, Xbal, and Xhol and T4 DNA ligase were from New England Biolabs.

### Example 2

### Transformation of Algal Cells

This example describes the transformation of one strain of Chlamydomonas reinhardtii and one strain of Chlorella zofengenisis with the recombinant molecules described in Examples by electroporation to introduce Oleo1 into the organisms.

The strains and culture conditions are as follows. Wild type C. reinhardtii cells of strain mt-[137c], were obtained from the Chlamydomonas Research Center (St. Paul, Minn.). Cells were grown at room temperature (22 C.) on a shaker at 120 rpm at a light intensity of 50 μE m⁻² s⁻¹ and a starting cell density of approximately 1.0 x 10⁵ cells/mL (FIG. 4). Cells were grown in Tris-Acetate-Phosphate (TAP) culture medium (Harris, 1989). Illumination was continuous at 50 μEm⁻² s⁻¹ from white LED panels. The flasks, wells, or microplates were agitated on a gyratory shaker (100 rpm) at 22° C. without aeration.

### Chlamydomonas Transformation

Electroporation of Chlamydomonas cells with plasmid DNA was performed as previously described (Invitrogen, 2013). Briefly, 2 μg plasmid DNA was mixed in a 4 mm electroporation cuvette with 5.4×10⁴ wild type C. reinhardtii cells in exponential growth, and incubated at 22° C. for 5 minutes. After incubation, plasmid DNA was electroporated into Chlamydomonas cells with the pulse generator set at 50 μF, 600 V, and resistance of infinity. After electroporation, cells were resuspended in 12 ml of TAP+40 mM sucrose and incubated for 24 hours at 22° C. under white LED panels of intensity 50 μE m⁻² s⁻¹ with agitation of 100 rpm. After recovery, cells were pelleted, washed with TAP+40 mM sucrose and resuspended in 750 μl TAP+40 mM sucrose. 250 μl of the cells were plated on each of three TAP+10 mg/L hygromycin agar plates and incubated at 22° C. under white LED lights of 50 μE m⁻² s⁻¹ for 5 days or until colonies are clearly visible.

### Chlorella Transformation

The C. zofingiensis strain CZUTEX032 was obtained from Dr. Henri Gerken at Arizona State University. The algae were cultured at 22° C. in CZM1 media consisting of, per litre: 0.5 g KNO₃, 0.62 g NaH₂PO₄ H₂O, 0.089 g Na₂HPO₄ H₂O, 0.247 g MgSO₄ 7H₂O, 14.7 mg CaCl₂ 2H₂O, 6.95 mg FeSO₄ 7H₂O, 0.061 mg H₃BO₃, 0.169 mg MnSO₄ H₂O, 0.287 mg ZnSO₄ 7H₂O, 0.0025 mg CuSO₄, 5H₂O, and 0.01235 mg (NH₄)₆MO₇O₂₄ 4H₂O. The pH of the media was adjusted to 6.8 before autoclaving. 5 mL of cells at density 1×10⁶ cells/mL were inoculated with 45 mL of CZM1 media in 125 mL flasks with continuous illumination of 50 μE m⁻² s⁻¹ and agitated at 100 rpm with no aeration. CZM1 agar plates were prepared using the above protocol, with 15 g/L agar. Electroporation was used to transform C. zofingiensis. The cells were grown in liquid CZM1 media until they reached a concentration of 5.0×10⁶ cells/mL. The cells were then pelleted (1200 g, 5 min), then resuspended and washed twice with ice-cold double-distilled water to reach a final concentration of 1×10⁸ cells/mL. For each transformation, 0.2 mL of algal cells were used with 1 μg of plasmid DNA and 2 μg of salmon sperm DNA. Electroporation was conducted using capacitance of 50 μF, resistance of 200 ohms, and field strength of 1.6 kV. To ensure successful transformation, cells and reagents were kept on ice for the entirety of the transformation protocol. The electroporated cells were then recovered for 24 hours in 10 mL liquid CZM1 media at 22° C. with continuous illumination of 50 μE m⁻² s⁻¹ and agitation of 100 rpm.

After the 24-hour recovery period, the electroporated cells were pelleted (1200 g, 5 min) and spread on CZM1 agar plates containing 0.5 μM norflurazon for selection. The plates were taped with micropore adhesive tape to allow for sufficient aeration and placed in an algal growth chamber with 1.5% CO₂ in air, with constant illumination of 50 μE m⁻² s⁻¹ at 26° C. The plates were re-taped and the collected condensation was pipetted off every three days. The colonies appearing after 14 days were picked. Each single colony was resuspended in 20 μL CZM1 and spotted onto new CZM1+0.5 μM norflurazon agar plates. Colonies appearing after 3 to 4 weeks were transferred and restreaked twice on new selection plates.

### Example 3

### Production of ALPs in Algal Cells

ALPs were produced in algal cells by inoculating 50 mL of TAP media at a density of 1×10⁵ cells/mL, and growing to late log phase. Cultures to be nitrogen stressed (to be grown in TAP-N) were pelleted (1200 g, 5 min) and resuspended into an equal volume TAP-N medium (Siaut 2011, BMC Biotechnology 2011 Jan 21; 11:7 doi 10.1186/1472-6750-11-7). Control cultures (to be grown in TAP+N) were pelleted (1200 g, 5 min) and resuspended in fresh TAP medium. All cultures were incubated 5 days after resuspension under standard growth conditions (50 μE m⁻² s⁻¹ from white LED panels, agitated at 100 rpm, at 22° C.) before imaging.

To image the ALPs produced in algal cells, 10 ul of the cultured cells of interest were transferred to a coated slide. The slide coat consisted of 2% agarose gel (Thermo Fisher Scientific) in TAP or TAP−N medium as appropriate and with the addition of 0.0001% w/v Nile Red (Sigma-Aldrich) when detection of triacylglycerides was desired. A 24×40 mm (No. 1 ½) cover glass (Corning) was placed on top of the sample before the slide was subjected to analysis. An Olympus Fluoview FV10i (Olympus Canada Inc., Richmond Hill, Ontario) laser scanning confocal microscope was used to observe and capture images of the cells. All images were captured using an Olympus UPlanSApo 60× oil immersion objective (Olympus Canada Inc., Richmond Hill, Ontario). Additional digital magnification of 10× (total magnification of 600×) was applied using the Fluoview FV10i 1.2a software. Laser excitation and emission wavelengths for cyan fluorescent protein (CFP) were set to 428 nm and 536 nm respectively. Yellow fluorescent protein (YFP) excitation and emission wavelengths were set to 480 nm and 527 nm respectively. Laser excitation and emission wavelengths for detection of triacylglycerides (TAGs) stained with Nile Red (NR) were set to 533 nm and 574 nm respectively. Where applicable, chloroplast autofluorescence (CHL) was imaged using an excitation wavelength of 473 nm and emission wavelength of 670 nm.

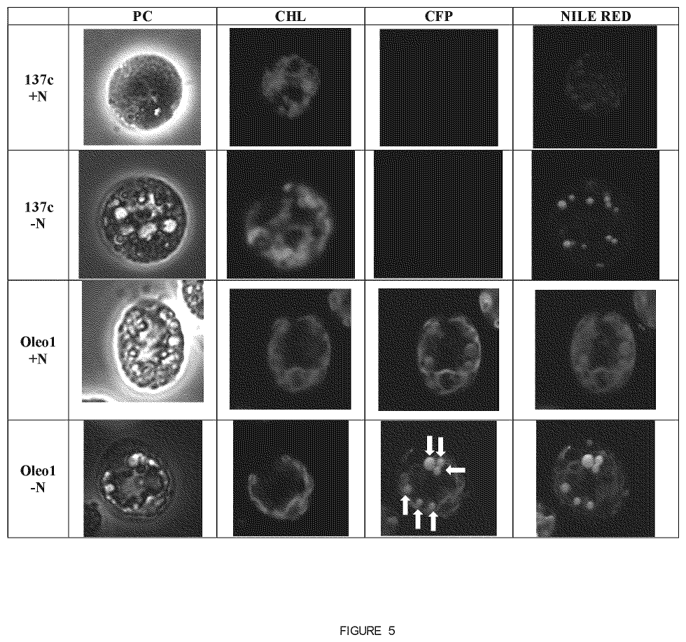

Referring to FIG. 5, Oleosin1-CFP is targeted to the chloroplast under homeostatic conditions while under stress (nitrogen depletion, TAP-N), Oleo1-CFP is targeted to the ALPs.

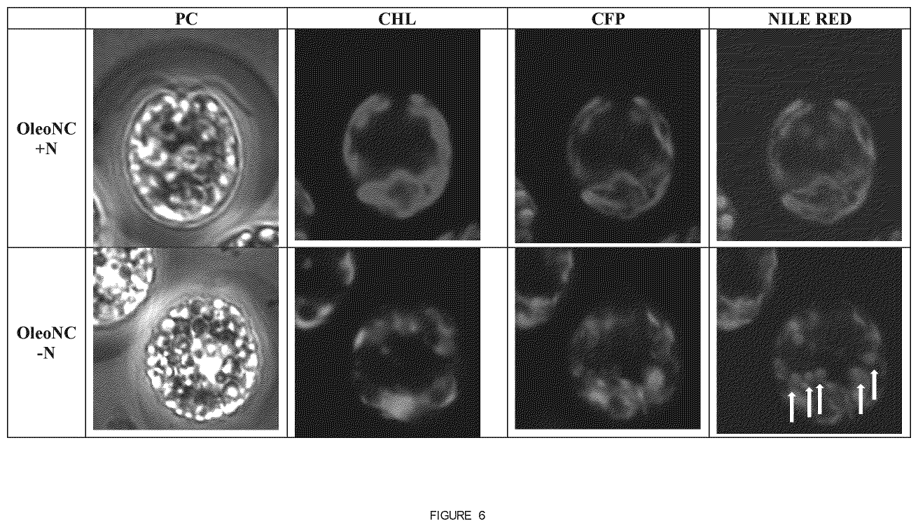

Referring to FIG. 6, when the hydrophobic region is deleted and the N and C lobe regions fused to form the OleoNC construct which is then fused to CFP, the genetic fusion is targeted to the chloroplast under homeostatic conditions and under non-homeostatic conditions it is targeted to the ALPs and chloroplast. It does not form ALPs in the same distinct form and quantity as Oleo1-CFP. In images in Nile Red column, white arrows indicate areas of Nile Red where CFP and Nile Red are both present but chloroplast autofluorescence is not present thus indicating presence of ALPs.

### Example 4

### Isolation of Lipids

Lipids were isolated from 500 mL cultures grown to late log phase in TAP medium, pelleted and resuspended into 500 mL of TAP-N medium and incubated for 5 days. Cultures were harvested by centrifugation (2000 g, 7 min), washed once with cold distilled water, and resuspended in 15 mL buffer (10 mM sodium phosphate pH 7.5; 1 mM EDTA; 10 mM KCl; 1 mM MgCl; 2 mM DTT and 0.6 M sucrose) (James 2010). Cells were homogenized 2-6 times at air pressure 40 psi (EmulsiFlex-C3) and the homogenate centrifuged (6000 g, 4 C, 120 min).

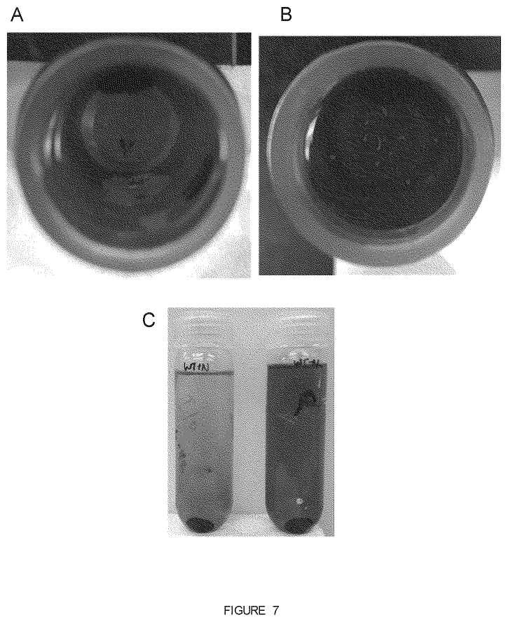

FIG. 7 depicts the lipids isolated from Chlamydomonas 137c cells grown in TAP and TAP-N media. Wild type cells grown in TAP yield no visible lipids when viewed from the top (7A) or side (7C, left side), and only a small amount of lipids are visible as pale flecks from a top view (7B) when extracted after growth in TAP-N. There is no visible lipid layer in the side view (7C, right side). Pale flecks on the surface in (7B) indicate lipids.

### Example 5

### Isolation of ALPs

ALPs were isolated from 500 mL cultures grown to late log phase in TAP medium, pelleted and resuspended into 500 mL of TAP-N medium and incubated for 5 days.

Cultures were harvested by centrifugation (2000 g, 7 min), washed once with cold distilled water, and resuspended in 15 mL buffer (10 mM sodium phosphate pH 7.5; 1 mM EDTA; 10 mM KCl; 1 mM MgCl; 2 mM DTT and 0.6 M sucrose) (James 2010). Cells were homogenized 2-6 times at air pressure 40 psi (EmulsiFlex-C3) and the homogenate centrifuged (6000 g, 4 C, 120 min).

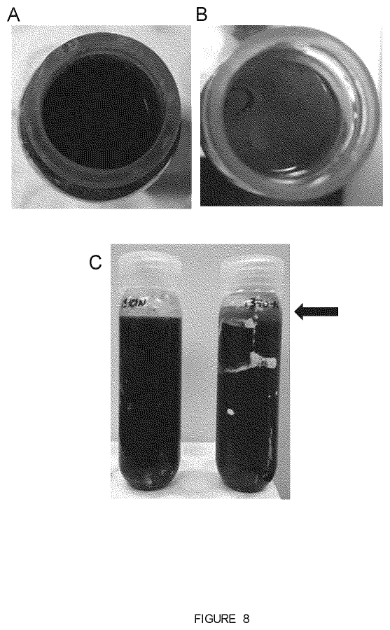

FIG. 8 depicts the lipids isolated from Oleo1 cells grown in TAP and TAP-N media. Oleo1 cells grown in TAP yield no visible ALPs when viewed from the top (8A) or side (8C, left side), however a layer of ALPs are visible from both top (8B) and side views (8C, right side) when extracted after growth in TAP-N. There is a thick visible ALP layer in the side view. Black arrow in FIG. 8 indicates ALPs.

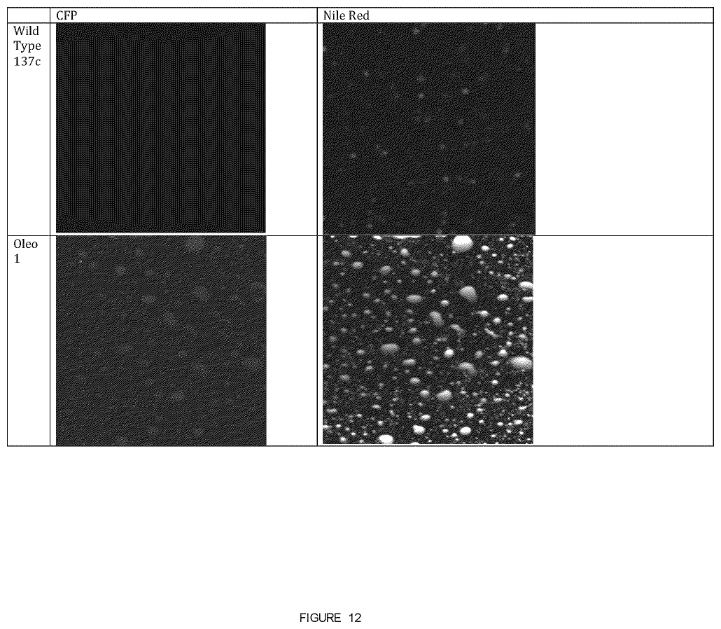

FIG. 12 depicts a comparison of the lipids isolated in Example 4 (Isolation of lipids) and Example 5 (Isolation of ALPs). 10 ul of lipids or ALPs was applied to a slide and imaged as described in Example 3. Images of 137c depict no CFP signal and a very small amount of lipid under Nile Red. Images of Oleo1 depict CFP signal, thus indicating the presence of the Oleosinl-CFP fusion protein. Under Nile Red, a large amount of lipids can be seen in the same location as CFP signal, indicating the presence of triacylglycerols and Oleosinl-CFP in the same location. This indicates the presence of ALPs.

### Example 6

### Expression of Recombinant Proteins in Association with ALPs

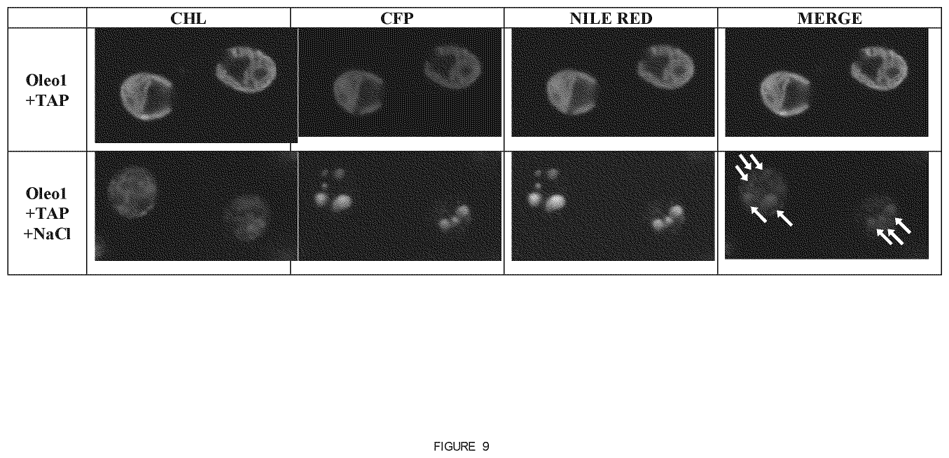

ALPs were produced in the strains 137c and Oleo1 of Chlamydomonas reinhardtii 137c under homeostatic growth conditions and non-homeostatic growth conditions. Homeostatic growth conditions were incubation at room temperature (22C) on a gyratory shaker at 100 rpm with continuous illumination of 50 μE m⁻² s⁻¹ from white LED panels. Non-homeostatic growth conditions were as in homeostatic growth conditions with the addition of sodium chloride to a final concentration of 100 mM to TAP medium, and further growth at 22° C. for 3 days at which point cells were imaged as described in Example 3 (FIG. 9).

In addition to the non-homeostatic conditions mentioned above, cultures were subjected to nitrogen deprivation (results in Example 3).

FIG. 9 depicts transgenic Chlamydomonas cells containing the Oleo1-CFP construct under homeostatic (TAP) and non-homeostatic (TAP+NaCl) conditions. Viewing conditions were chloroplast autofluorescence (CHL), CFP, and Nile Red. In transgenic Oleo1-CFP cells under homeostatic conditions, Oleosinl-CFP is targeted to the chloroplast. This is indicated by the presence of CFP and chlorophyll autofluorescence in the same areas of the cell. Under non-homeostatic conditions of TAP+100 mM NaCl (TAP+NaCl), Oleo1-CFP is targeted to the ALPs. ALPs are indicated by the colocalization of Nile Red staining for triacylglycerides and CFP. In images in “merge” column, white arrows indicate areas of only CFP where chloroplast autofluorescence is not present. This indicates targeting specifically to ALPs.

### Example 7

### Production of ALPs in Chlorella

ALPs were produced in chlorella by inoculating 50 mL of CZM1 at a density of 1×10⁵ cells/mL, and growing to late log phase. Cultures to be stressed were allowed to continue to grow. Control cultures (to be grown in TAP+N) were pelleted (1200 g, 5 min) and resuspended in fresh CZM1 medium. All cultures were incubated an additional three weeks under standard growth conditions (50 μE m⁻² s⁻¹ from white LED panels, agitated at 100 rpm, at 22° C.) before imaging.

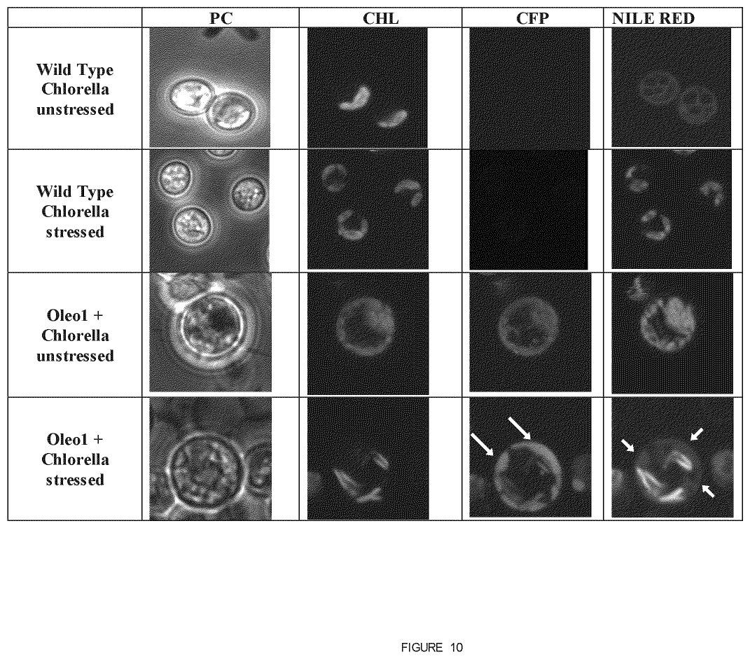

FIG. 10 depicts wild type (WT) and transgenic Chlorella cells containing the Oleo1-CFP construct under homeostatic (CZM1) media) and non-homeostatic (TAP−N media) conditions. In transgenic Oleo1-CFP cells under homeostatic conditions, Oleosinl-CFP is targeted to the chloroplast. This is indicated by the presence of CFP and chlorophyll autofluorescence in the same areas of the cell. Under stress, Oleo1-CFP is targeted to the ALPs. This is indicated by areas of CFP and NR signal as indicated by white arrows in the CFP and NR columns.

### Example 8

### Isolation of Chloroplasts

Chloroplasts were isolated as described in Mason, et al. (2006). Nat. Protoc. 1, 2227-2230 with some modifications. Chlamydomonas cells used were 137c or Oleo1 transgenic strain, and cultures were grown under 24 h 50 μE m⁻² s⁻¹ light. Cells were allowed to sit in isolation buffer for 10 minutes before being passed through the 27-gauge needle at 0.2 ml s⁻¹. Cells were imaged immediately after isolation from the 45-65% interface and washing in isolation buffer +10% (w/v) BSA.

### Example 9

### Oleosin2, Oleosin3, and Oleosin 4 Constructs

Using the Oleo1 construct as a template, Oleosin1 was removed and replaced with one of Oleosin2, Oleosin3, and Oleosin 4 in the pChlamy_3 construct. When necessary, sequences were sent to Genscript Biotech for optimization of expression in algae prior to cloning.

Following methods described earlier, each of these constructs was electroporated into 137c and examined for ALP production.

To study ALP production in algae with more than one oleosin, the HygR gene in the modified pChlamy_3 (FIG. 2) was excised and replaced with the PDS gene encoding norflurazon resistance that had been optimized for expression in Chlamydomonas. Stable transgenic lines of Oleo2, Oleo3, and Oleo4 were transformed with this Oleo1 as described earlier, but plated on TAP+10 mg/L hygromycin +2 uM norflurazon agar plates and incubated at 22° C. under white LED lights of 50 uE m⁻² s⁻¹ for ten days. Colonies that appeared were resuspended in 10 uL liquid TAP and spotted on fresh TAP+10 mg/L hygromycin+2 uM norflurazon agar plates. After an additional week, colonies were picked and used to inoculate liquid TAP media containing 10 mg/L hygromycin+2 uM norflurazon, and incubated at 22° C. under white LED lights of 50 uE m⁻¹ s⁻¹ on a gyratory shaker set at 100 rpm until used for further study.

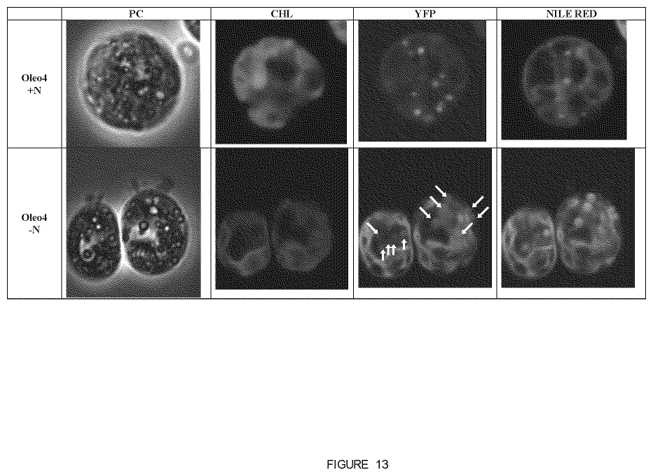

FIG. 13 depicts transgenic Chlamydomonas cells containing the Oleo4-YFP construct under homeostatic (+N) and non-homeostatic (−N) conditions. Viewing conditions were phase contrast (PC), chloroplast autofluorescence (CHL), Yellow Fluorescent Protein (YFP), and Nile Red. In Oleo4-YFP cells under homeostatic conditions, Oleosin4-YFP is targeted to the chloroplast, with small points of high intensity within the chloroplast. This is indicated by the presence of YFP and CHL in the same areas of the cell. Under stress (−N), Oleosin4-YFP is targeted to the ALPs. ALPs are indicated by the presence of Nile Red (triacylglycerides) and YFP in the same location. In images in YFP column, white arrows indicate areas of YFP which are also present in the Nile Red column and absent in the CHL column. This indicates the presence of ALPs.

Findings summarized in Table 1 below.

| Strain | Targeting and Expression of ALPs |
|---|---|
| Oleo1 | targets to chloroplast, forms ALPs when stressed |
| Oleo2 | targets to chloroplast, does not form ALPs when |
| | stressed |
| Oleo3 | targets to chloroplast, forms ALPs when stressed |
| Oleo4 | targets to chloroplast, forms ALPs when stressed |
| Oleo1 + Oleo2 | targets to chloroplast, does not form ALPs when |
| | stressed |
| Oleo1 + Oleo3 | targets to chloroplast, forms ALPs when stressed |
| Oleo1 + Oleo4 | targets to chloroplast, forms ALPs when stressed |

TABLE 2
Oleosin Protein Sequences

| SEQ ID | | |
|---|---|---|
| NO | SEQUENCE | Organism |
| 1 | MADTARGTHHDIIGRDQYPMMGRDRDQYQ | Arabidopsis thaliana |
| | MSGRGSDYSKSRQIAKAATAVTAGGSLLVLS | Oleosin |
| | SLTLVGTVIALTVATPLLVIFSPILVPALITVALLI | |
| | TGFLSSGGFGIAAITVFSWIYKYATGEHPQGS | |
| | DKLDSARMKLGSKAQDLKDRAQYYGQQHT | |
| | GGEHDRDRTRGGQHT | |
| 2 | MATATDRAPHQVQVHTPTTQRVDVPRRGYD | Arachis hypogaea |
| | VSGGGIKTLLPERGPSTSQIIAVLVGVPTGGT | Oleosin |
| | LLLLSGLSLLGTIIGLAIATPVFIFFSPVIVPAVV | |
| | TIGLAVTGILTAGACGLTGLMSLSWMINFIRQ | |
| | VHGTTVPDQLDSVKRRMADMADYVGQKTK | |
| | DAGQEIQTKAQDVKRSSS | |
| 3 | MAEHYGQQQQTRAPHLQLQPRAQRVVKAA | Sesamum indicum |
| | TAVTAGGSLLVLSGLTLAGTVIALTIATPLLVIF | Oleosin |
| | SPVLVPAVITIFLLGAGFLASGGFGVAALSVL | |
| | SWIYRYLTGKHPPGADQLESAKTKLASKARE | |
| | MKDRAEQFSQQPVAGSQTS | |
| 4 | MQKIHDHTPNPTQILGFITLFVSGAVLLFLTGL | Pinus massoniana |
| | TLTGTVVGLVVLTPVLIFFSPILIPLATVLFVAV | Oleosin |
| | AGFLSAGGFGLAALSAISWLYNYIKGRHPPG | |
| | ADQIDYARMRIADTATHVKDYAREYGGYLQS | |
| | KIQDAAPGA | |
| 5 | MANQTRTHQDIIVRDSRSTLDRDHPKTGAQ | Brassica napus |
| | MVKVATGVAAGGSLLVLSGLTLAGTVIALAVA | Oleosin |
| | TPLLIIFSPVLVPAVITVVLIITGFLASGGFGIAAI | |
| | TAFSWLYRHMTGSGSDQKIESARMKVGSRG | |
| | YDTKSGQHNIGVHQQHQQAAS | |
| 6 | MADHHRGATGGGGGYGDLQRGGGMHGEA | Zea mays |
| | QQQQKQGAMMTALKAATAATFGGSMLVLS | Oleosin |
| | GLILAGTVIALTVATPVLVIFSPVLVPAAIALAL | |
| | MAAGFVTSGGLGVAALSVFSWMYKYLTGKH | |
| | PPAADQLDHAKARLASKARDVKDAAQHRID | |
| | QAQGS | |
| 34 | MADTHRVDRTDRHFQFQSPYEGGRGQGQY | Arabidopsis thaliana |
| | EGDRGYGGGGYKSMMPESGPSSTQVLSLLI | Oleosin 2 |
| | GVPVVGSLLALAGLLLAGSVIGLMVALPLFLL | |
| | FSPVIVPAALTIGLAMTGFLASGMFGLTGLSSI | |
| | SWVMNYLRGTRRTVPEQLEYAKRRMADAV | |
| | GYAGQKGKEMGQHVQNKAQDVKQYDISKP | |
| | HDTTTKGHETQGRTTAA | |
| 35 | MADQTRTHHEMISRDSTQEAHPKARQMVKA | Arabidopsis thaliana |
| | ATAVTAGGSLLVLSGLTLAGTVIALTVATPLLV | Oleosin 3 |
| | IFSPVLVPAVVTVALIITGFLASGGFGIAAITAF | |
| | SWLYRHMTGSGSDKIENARMKVGSRVQDTK | |
| | YGQHNIGVQHQQVS | |
| 36 | MANVDRDRRVHVDRTDKRVHQPNYEDDVG | Arabidopsis thaliana |
| | FGGYGGYGAGSDYKSRGPSTNQILALIAGVP | Oleosin 4 |
| | IGGTLLTLAGLTLAGSVIGLLVSIPLFLLFSPVI | |
| | VPAALTIGLAVTGILASGLFGLTGLSSVSWVL | |
| | NYLRGTSDTVPEQLDYAKRRMADAVGYAGM | |
| | KGKEMGQYVQDKAHEARETEFMTETHEPGK | |
| | ARRGS | |

TABLE 3
Oleosin Nucleotide Sequences

| SEQ ID | | |
|---|---|---|
| NO | SEQUENCE | Organism |
| 7 | ATGGCGGATACAGCTAGAGGAACCCATCA | Arabidopsis thaliana |
| | CGATATCATCGGCAGAGACCAGTACCCGA | |
| | TGATGGGCCGAGACCGAGACCAGTACCAG | |
| | ATGTCCGGACGAGGATCTGACTACTCCAA | |
| | GTCTAGGCAGATTGCTAAAGCTGCAACTGC | |
| | TGTCACAGCTGGTGGTTCCCTCCTTGTTCT | |
| | CTCCAGCCTTACCCTTGTTGGAACTGTCAT | |
| | AGCTTTGACTGTTGCAACACCTCTGCTCGT | |
| | TATCTTCAGCCCAATCCTTGTCCCGGCTCT | |
| | CATCACAGTTGCACTCCTCATCACCGGTTT | |
| | TCTTTCCTCTGGAGGGTTTGGCATTGCCGC | |
| | TATAACCGTTTTCTCTTGGATTTACAAGTAC | |
| | GCAACGGGAGAGCACCCACAGGGATCAGA | |
| | CAAGTTGGACAGTGCAAGGATGAAGTTGG | |
| | GAAGCAAAGCTCAGGATCTGAAAGACAGA | |
| | GCTCAGTACTACGGACAGCAACATACTGGT | |
| | GGGGAACATGACCGTGACCGTACTCGTGG | |
| | TGGCCAGCACACT | |
| 8 | ATGGCTACTGCTACTGATCGTGCACCTCA | Arachis hypogaea |
| | CCAGGTTCAAGTTCACACCCCCACCACA | |
| | CAACGCGTCGACGTTCCACGCCGCGGCT | |
| | ACGATGTTAGTGGTGGTGGTATTAAGACT | |
| | CTTCTCCCCGAGAGAGGTCCGTCCACCT | |
| | CTCAAATCATCGCCGTCCTCGTCGGCGT | |
| | CCCCACTGGGGGCACTCTGTTGCTCCTC | |
| | TCCGGCCTTTCACTTCTCGGAACCATAAT | |
| | CGGGCTGGCAATTGCCACCCCGGTTTTT | |
| | ATCTTCTTCAGCCCGGTTATAGTTCCCGC | |
| | GGTCGTTACCATTGGACTTGCGGTCACT | |
| | GGTATTCTCACGGCGGGAGCATGTGGAC | |
| | TAACCGGGCTGATGTCTTTGTCATGGATG | |
| | ATTAACTTCATCCGACAGGTACATGGGAC | |
| | GACGGTGCCGGATCAGCTGGACTCAGTG | |
| | AAGCGGCGCATGGCGGACATGGCGGATT | |
| | ACGTGGGGCAGAAGACAAAGGATGCTGG | |
| | CCAAGAGATACAGACTAAGGCCCAGGAT | |
| | GTTAAGAGGTCATCATCATAA | |
| 9 | ATGGCTGAGCATTATGGTCAACAACAGCAG | Sesamum indicum |
| | ACCAGGGCGCCTCACCTGCAGCTGCAGCC | |
| | GCGCGCCCAGCGGGTAGTGAAGGCGGCC | |
| | ACCGCCGTGACAGCCGGCGGCTCGCTTCT | |
| | CGTCCTCTCTGGCCTCACATTAGCCGGAA | |
| | CTGTTATTGCGCTCACCATCGCCACTCCGC | |
| | TGCTTGTGATCTTTAGCCCCGTTCTGGTGC | |
| | CGGCGGTCATAACCATTTTCTTGCTGGGTG | |
| | CGGGTTTTCTGGCATCCGGAGGCTTCGGC | |
| | GTGGCGGCGCTGAGTGTGCTGTCGTGGAT | |
| | TTACAGATATCTGACAGGGAAACACCCGCC | |
| | GGGGGCGGATCAGCTGGAATCGGCAAAGA | |
| | CGAAGCTGGCGAGCAAGGCGCGAGAGAT | |
| | GAAGGATAGGGCAGAGCAGTTCTCGCAGC | |
| | AGCCTGTTGCGGGGTCTCAAACTTCTTGA | |
| 10 | ATAGGGCAAGCAGTGGTATCAACGCAGAG | Pinus massoniana |
| | TACATGGGAAGTTAGCCTCTGCAACGCTAA | |
| | AATTAAGTTCATACAGCGAGTAGTTTACAG | |
| | GTTTTTTTTTGCTTTTCAGGGTTTTTCAGGG | |
| | TTTTTAAGCATCATTTGAGATGGCTGATCA | |
| | GTTCACGAAGTGATGCAGAAAATTCATGAT | |
| | CATACGCCCAATCCAACTCAGATCTTGGGT | |
| | TTCATCACCCTGTTCGTTTCTGGTGCAGTT | |
| | TTGCTGTTTTTGACTGGTCTCACATTAACTG | |
| | GAACGGTTGTAGGGTTGGTGGTTCTCACT | |
| | CCTGTTCTCATTTTCTTCAGTCCCATATTGA | |
| | TACCATTAGCAACTGTGCTGTTTGTAGCAG | |
| | TTGCAGGGTTCTTGTCTGCAGGAGGCTTTG | |
| | GACTGGCAGCCTTATCTGCCATTTCATGGC | |
| | TTTACAACTACATTAAGGGCAGGCATCCCC | |
| | CTGGGGCAGACCAGATAGACTATGCCCGC | |
| | ATGCGTATTGCAGATACAGCGACCCATGTT | |
| | AAGGATTATGCCCGTGAATATGGTGGGTAT | |
| | TTGCAGAGCAAGATTCAGGATGCTGCTCC | |
| | CGGAGCTTAAGTAAGGTCTTGGACCGTAAT | |
| | AAATTCAGGATATATGCAGTATGTATATGCT | |
| | CTCATTTAGCTGCTCATCTGATTTCCATGG | |
| | GGTGAATCAGTTGTTTTGCAGTACGTGGG | |
| | GGTCTGTTTATTTTGTGAGTTGCATCATTTT | |
| | CTTTATGGTGAAGTTCTTTTTGTTGTTGTTT | |
| | TTTTTT | |
| 11 | GGTGTCGGAGAGATCATAACAAGAATCAAT | Brassica napus |
| | GGCGAATCAAACAAGAACCCATCAAGACAT | |
| | AATAGTCCGAGACAGTAGAAGTACCCTAGA | |
| | CAGAGACCATCCGAAGACAGGGGCGCAGA | |
| | TGGTGAAGGTAGCGACTGGTGTCGCAGCC | |
| | GGTGGATCCCTCCTAGTCCTCTCAGGCTTA | |
| | ACACTCGCCGGTACAGTGATAGCACTCGC | |
| | TGTAGCCACTCCTCTGCTTATTATATTTAGC | |
| | CCTGTCTTGGTCCCAGCGGTGATCACCGT | |
| | GGTTCTCATCATCACTGGGTTCCTTGCCTC | |
| | CGGTGGCTTTGGTATAGCCGCCATAACCG | |
| | CCTTCTCTTGGCTCTACAGGCACATGACGG | |
| | GGTCTGGATCGGATCAAAAGATAGAGAGT | |
| | GCTCGAATGAAGGTGGGAAGTAGAGGGTA | |
| | TGATACCAAGTCTGGGCAGCACAACATTG | |
| | GAGTCCATCAGCAACACCAACAAGCAGCTT | |
| | CTTAAAATTAATGTCAGATAAAACTATTTCC | |
| | ACACAAATTGTCATATCCATCGTTTGATCTC | |
| | TTTTCTTCGTTTTCTCACTGCTTGTGTTGTA | |
| | ACGTAACGAGTAATAATAACATGAGTGTAT | |
| | GTTGCCCAAAAAAAAAAAAAAAAAAAAAAA | |
| | AAAAA | |
| 12 | CGGGGTGGCGGGGGCTACGGCGACCTCC | Zea mays |
| | AGCGCGGGGGCGGCATGCACGGCGAGGC | |
| | GCAGCAGCAGCAGAAGCAGGGCGCCATG | |
| | ATGACGGCGCTCAAGGCCGCGACGGCCG | |
| | CGACCTTCGGCGGGTCGATGCTGGTGCTG | |
| | TCCGGGCTGATCCTGGCCGGCACCGTGAT | |
| | CGCGCTCACGGTGGCCACCCCCGTGCTG | |
| | GTGATCTTCAGCCCGGTGCTGGTGCCAGC | |
| | CGCCATCGCGCTGGCGCTCATGGCGGCC | |
| | GGGTTCGTCACCTCCGGCGGCCTCGGCGT | |
| | CGCTGCGCTGTCCGTGTTCTCCTGGATGT | |
| | ACAAGTACCTGACGGGCAAGCACCCGCCG | |
| | GGCGCCGACCAGCTGGACCACGCCAAGG | |
| | CGAGGCTGGCGTCCAAGGCCCGCGACATC | |
| | AAGGATGCAGCACAGCACCGCATCGACCA | |
| | GGCGCAGGGGTCTTGAGAGAAGAACCACA | |
| | CTCGAGCGGACCGCGCGCGCGTCCTCCTT | |
| | GAACCACTGCCGGCGCGGCGGCATATGG | |
| | CCCTTAAAGGCGGTGGCTGCTGCTACGTA | |
| | CGCTGCCGTAGAGTCTCGGTCGCCGCGAT | |
| | AGCTCTAGCTAGTCGTTTATGTGTTGTGCT | |
| | TTGTGTGTGCATGCATGTGTCTGGGGGCA | |
| | TGCAGTCAGTGCAGTACTATATGCTGTATG | |
| | CGTCTCTCTTTGATCGGAGAGGCGGATGT | |
| | ACAGCATGCTCGATATGTCTAGTTTGGATG | |
| | TCATGTTTATGATGAGGAATAAAATGCAGG | |
| | TCAGGTG | |
| 37 | ATTACAAAGAAAATAGGTAAAAACAATTTCT | Arabidopsis thaliana |
| | CATTAGCTTACAATGGCGGATACACACCGT | Oleosin 2 |
| | GTCGACCGTACTGATAGACACTTTCAATTT | |
| | CAGTCGCCCTATGAAGGCGGCCGAGGTCA | |
| | AGGTCAGTATGAAGGTGACCGTGGTTACG | |
| | GTGGTGGCGGTTACAAGAGCATGATGCCT | |
| | GAAAGTGGCCCATCTAGTACCCAAGTATTG | |
| | TCCCTGTTGATTGGAGTCCCTGTCGTCGGT | |
| | TCGCTACTTGCCTTGGCTGGATTACTTCTA | |
| | GCTGGTTCGGTGATCGGCTTAATGGTTGCT | |
| | TTACCACTATTTCTCCTCTTCAGCCCGGTT | |
| | ATAGTCCCAGCGGCTCTAACTATCGGGCTT | |
| | GCAATGACAGGCTTTTTAGCCTCGGGGAT | |
| | GTTCGGTCTAACCGGGCTTAGCTCAATCTC | |
| | ATGGGTCATGAACTATCTTCGTGGGACAAG | |
| | GAGAACTGTGCCTGAGCAATTGGAGTATG | |
| | CTAAGAGGAGAATGGCTGATGCGGTTGGC | |
| | TACGCAGGACAAAAGGGCAAAGAAATGGG | |
| | CCAGCATGTGCAGAACAAGGCCCAAGATG | |
| | TTAAACAATATGATATTTCTAAGCCACATGA | |
| | CACTACCACTAAGGGTCATGAGACTCAGG | |
| | GGAGGACGACGGCTGCATGATGAGTTTTC | |
| | AGTATGAACGGTAGATATGTGTTTTCACTA | |
| | TTATGTCGTTTTTTCTGCATTTTCAATATGA | |
| | TGTTATGTGTTTTTTTTGTTTGGCTTTTTGTT | |
| | GAACCGTGTATGTGTTTTATGTTTTTGTAAG | |
| | CATGAAAGATCGCAAGTGTTGTGGTAATAT | |
| | TTGAATGTAATAATATGATAAGTTGATAAAT | |
| | CATGGGAACATTTAAATTAGGTGGACATGT | |
| | TTAGCTATTTGATACTCACAGTTCTTTAGGT | |
| | TTCTAGTTTTTTTTTTGTCTATATAGA | |
| 38 | CCTTCATAAATCATCATTACTTACTCCGAAA | Arabidopsis thaliana |
| | ACACATCACTACCTTTTCTCTGCTAATTTCT | Oleosin 3 |
| | ACTATTTGTGTTGGAGGGATATATTATAATA | |
| | AAAAGAACCAATGGCGGACCAAACAAGAA | |
| | CCCATCACGAGATGATAAGCCGAGACAGT | |
| | ACCCAAGAGGCCCATCCGAAGGCCAGGCA | |
| | GATGGTGAAGGCAGCAACCGCTGTCACAG | |
| | CCGGTGGATCCCTACTTGTCCTCTCCGGC | |
| | TTAACACTCGCTGGAACAGTCATCGCACTC | |
| | ACGGTGGCTACTCCTCTCCTCGTCATCTTC | |
| | AGCCCCGTCTTGGTTCCAGCAGTGGTAAC | |
| | CGTTGCTCTCATCATTACCGGATTCCTTGC | |
| | ATCCGGTGGCTTTGGAATAGCCGCCATTAC | |
| | CGCCTTCTCTTGGCTCTACAGGCACATGAC | |
| | GGGATCTGGATCGGATAAGATAGAGAATG | |
| | CTCGGATGAAGGTTGGAAGCAGAGTGCAG | |
| | GATACTAAGTATGGGCAGCACAACATTGGA | |
| | GTCCAACACCAACAAGTTTCTTAAAATTCAA | |
| | AGGCTAAAAACGATTTCCACATTAAATTGT | |
| | CATCCATCGTTAGATCTCTTCTCTTTGTTTT | |
| | TATTTTCAACGTTTATGTTGTAATGTGTGAT | |
| | ATATAATAACATGCAATGTATGATGTAATCT | |
| | TGTTGTTGTTGTATGAGTAATGTTTTGATCC | |
| | TTCTATGCGAGCGGAAATCTTCATATAGAC | |
| | AGTGTCATTGGTACATTACACAAATCTTCAT | |
| | AATCTCCTTCTACGCT | |
| 39 | CATCATCCTACATTCATACCTAAGCTAGCA | Arabidopsis thaliana |
| | AAGCAAACTACTAAAAGGGTCGTCAACGAC | Oleosin 4 |
| | AAGTTATTTGCTAGTTGGTGCATACTACAC | |
| | ACGGCTACGGCAACATTAAGTAACACATTA | |
| | AGAGGTGTTTTCTTAATGTAGTATGGTAATT | |
| | ATATTTATTTCGAAACTTGGATTAGATATAA | |
| | AGGTACAGTTAGTGAAAATATTTGGTTAGC | |
| | GGGTTGAATTAACCGGATATAGGAGTCATA | |
| | TATACAACTGTGAAAAAAGGATAAATACAA | |
| | AAAGGGAAGATGTTTTTCCGACACACAAGC | |
| | TAATTAAGTGCATCGAGAGGAGAGCAATTG | |
| | TAAAATGAATGTTTTGTTTGTTTTTGTACGG | |
| | TGGAGAGAAGAACGAAAAGATGATCAGGT | |
| | AAAAAATGAAACTTGGAAATCATGCAAAGC | |
| | CACACCTCTCCCTTCAACACATTCTTACGT | |
| | GTCGTCTTCTCTTCACTCCATATCTCCTTTT | |
| | TATTACCAACAAATATATTTCAATCCCATTT | |
| | ATATGTACGTTCTCGTAGACTTATCTCTATA | |
| | TACCCCCTTTAATTTGTTTGCTCTTAGCCTT | |
| | TACTTTATAGTTTTATATCATATCAATCGAC | |
| | ATGGCGAATGTGGATCGTGATCGGCGTGT | |
| | GCATGTAGACCGTACTGACAAACGTGTTCA | |
| | TCAGCCAAACTACGAAGATGATGTCGGTTT | |
| | TGGTGGCTATGGCGGTTATGGTGCTGGTT | |
| | CTGATTATAAGAGTCGCGGCCCCTCCACTA | |
| | ACCAAATCTTGGCACTTATAGCAGGAGTTC | |
| | CCATTGGTGGCACACTGCTAACCCTAGCT | |
| | GGACTCACTCTAGCCGGTTCGGTGATCGG | |
| | CTTGCTAGTCTCCATACCCCTCTTCCTCCT | |
| | CTTCAGTCCGGTGATAGTCCCGGCGGCTC | |
| | TCACTATTGGGCTTGCTGTGACGGGAATCT | |
| | TGGCTTCTGGTTTGTTTGGGTTGACGGGTC | |
| | TGAGCTCGGTCTCGTGGGTCCTCAACTAC | |
| | CTCCGTGGGACGAGTGATACAGTGCCAGA | |
| | GCAATTGGACTACGCTAAACGGCGTATGG | |
| | CTGATGCGGTAGGCTATGCTGGTATGAAG | |
| | GGAAAAGAGATGGGTCAGTATGTGCAAGA | |
| | TAAGGCTCATGAGGCTCGTGAGACTGAGT | |
| | TCATGACTGAGACCCATGAGCCGGGTAAG | |
| | GCCAGGAGAGGCTCATAAGCTAATATAAAT | |
| | TGCGGGAGTCAGTTGGAAACGCGATAAAT | |
| | GTAGTTTTACTTTTATGTCCCAGTTTCTTTC | |
| | CTCTTTTAAGAATATCTTTGTCTATATATGT | |
| | GTTCGTTCGTTTTGTCTTGTCCAAATAAAAA | |
| | TCCTTGTTAGTGAAATAAGAAATGAAATAAA | |
| | TATGTTTTCTTTTTTGAGATAACCAGAAATC | |
| | TCATAC | |

1. A method of producing a recombinant polypeptide in an algal cell, the method comprising:
growing algal cells comprising a recombinant polypeptide under homeostatic conditions to target the recombinant polypeptide to the algal chloroplast; wherein the recombinant polypeptide is a fusion polypeptide comprising an oleosin protein or fragment thereof and a protein of interest;
and isolating the recombinant protein.

2. The method of claim 1, comprising isolating the algal chloroplasts and wherein the recombinant protein is isolated from the isolated algal chloroplasts.

3. The method of claim 1, comprising subjecting the growing algal cells to non-homeostatic conditions to form accumulated lipid particles within the algal cells, wherein the accumulated lipid particles comprise the fusion polypeptide.

4. The method of claim 3, comprising isolating the accumulated lipid particles and wherein the recombinant protein is isolated from the isolated accumulated lipid particles.

5. The method of claim 1, comprising the step of introducing a nucleic acid encoding the recombinant polypeptide into the algal cell.

6. The method of claim 5, wherein the nucleic acid encoding the fusion polypeptide comprises one or more algal cell control elements.

7. The method of claim 1, wherein the oleosin protein or fragment thereof is a Oleo1 protein, Oleo2 protein, Oleo3 protein, Oleo 4 protein, derivatives or fragments or combinations thereof.

8. The method of claim 7, wherein the oleosin is a substantially the full length oleosin or an oleosin that excludes the hydrophobic domain, optionally selected from the group consisting of Oleo1 excluding the hydrophobic domain, Oleo2 excluding the hydrophobic domain, Oleo3 excluding the hydrophobic domain and Oleo4 excluding the hydrophobic domain.

9-10. (canceled)

11. The method of claim 3, wherein non-homeostatic conditions include a deficiency in one or more nutrients optionally nitrogen or phosphorus or an exogenous stress factor optionally selected from the group consisting of a non-homeostatic pH, a non-homeostatic salinity, and a non-homeostatic light intensity.

12-14. (canceled)

15. The method according to claim 1 wherein the oleosin is a protein encoded by a nucleic acid sequence having the sequence set forth in SEQ.ID NO: 7 to SEQ.ID NO: 12.

16. The method of claim 1 wherein the algal cell is selected form the group of algal cells consisting of green algae (Chlorophyceae), diatoms (Bacillariophyceae), yellow-green algae (Xanthophyceae), golden algae (Chrysophyceae), red algae (Rhodophyceae), brown algae (Phaeophyceae), dinoflagellates (Dinophyceae) or pico-plankton (Prasinophyceae and Eustigmatophyceae), wherein the green algae optionally belongs to the genus Clamydomonas, or Chlorella.

17. (canceled)

18. A method of producing algal chloroplasts enriched for recombinant polypeptide, the method comprising:
(a) introducing a nucleic acid into algal cells, the nucleic acid comprising as operably linked components
(i) a nucleic acid encoding a fusion polypeptide comprising an oleosin protein or fragment thereof to provide targeting to the algal chloroplast and a polypeptide of interest; and
(ii) a nucleic acid sequence capable of controlling expression in an algal cell;
(b) subjecting the algal cells in a growth medium to homeostatic conditions to target the fusion polypeptide to the algal chloroplast; and
(c) optionally isolating the algal chloroplasts.

19-30. (canceled)

31. A method for producing accumulated lipid particles, the method comprising:
(a) introducing a nucleic acid into algal cells, the nucleic acid comprising as operably linked components
a nucleic acid encoding a fusion polypeptide comprising an oleosin protein or fragment thereof to provide targeting to the algal chloroplast and a polypeptide of interest; and
a nucleic acid sequence capable of controlling expression in an algal cell;
(b) subjecting the algal cell to non-homeostatic conditions to form accumulated lipid particles within the algal cell and optionally isolating the accumulated lipid particles.

32. The method according to claim 31 wherein the algal cell is grown in a growth medium comprising quantities of one or more nutrients which are insufficient for homeostatic algal cell growth, wherein optionally the nutrients are nitrogen or phosphorus.

33. The method according to claim 31 wherein the algal cell while grown in the growth medium is subjected to an exogenous stress factor, optionally selected from the group consisting of a non-homeostatic pH, a non-homeostatic salinity, and a non-homeostatic light intensity.

34-35. (canceled)

36. The method according to claims 31, wherein the algal cell during a first time period is grown under homeostatic growth conditions wherein during such first time period substantially no accumulated lipid particles are formed, and wherein the algal cell during a second time period is grown under non-homeostatic growth conditions.

37. The method according to claim 36 wherein during the first period the algal cell is grown to logarithmic growth phase, and wherein during logarithmic phase the cells are subjected to non-homeostatic growth conditions to grow the cell under non-homeostatic conditions during the second time period.

38. The method according to claim 36 wherein during the first period the algal cell is grown to early stationary phase, and wherein during early stationary phase the cells are subjected to non-homeostatic growth conditions to grow the cell under non-homeostatic conditions during the second time period.

39. (canceled)

40. The method according to claim 31 wherein the oleosin is a protein encoded by a nucleic acid sequence having the sequence set forth in SEQ.ID NO: 7 to SEQ.ID NO: 12.

41. The method according to claim 31 wherein the algal cell is selected form the group of algal cells consisting of green algae (Chlorophyceae), diatoms (Bacillariophyceae), yellow-green algae (Xanthophyceae), golden algae (Chrysophyceae), red algae (Rhodophyceae), brown algae (Phaeophyceae), dinoflagellates (Dinophyceae) or pico-plankton (Prasinophyceae and Eustigmatophyceae), wherein the green algae optionally belongs to the genus Clamydomonas, or Chlorella.

42-47. (canceled)


SEQUENCE LISTING (39 sequences)

SEQ ID NO: 1 - protein, 172 aa, Arabidopsis thaliana
MADTARGTHHDIIGRDQYPMMGRDRDQYQMSGRGSDYSKSRQIAKAATAVTAGGSLLVLSSLTLVGTVIALTVATPLLVIFSPILVPALITVALLITGFLSSGGFGIAAITVFSWIYKYATGEHPQGSDKLDSARMKLGSKAQDLKDRAQYYGQQHTGGEHDRDRTRGGQHT

SEQ ID NO: 2 - protein, 176 aa, Arachis hypogaea
MATATDRAPHQVQVHTPTTQRVDVPRRGYDVSGGGIKTLLPERGPSTSQIIAVLVGVPTGGTLLLLSGLSLLGTIIGLAIATPVFIFFSPVIVPAVVTIGLAVTGILTAGACGLTGLMSLSWMINFIRQVHGTTVPDQLDSVKRRMADMADYVGQKTKDAGQEIQTKAQDVKRSSS

SEQ ID NO: 3 - protein, 145 aa, Sesamum indicum
MAEHYGQQQQTRAPHLQLQPRAQRVVKAATAVTAGGSLLVLSGLTLAGTVIALTIATPLLVIFSPVLVPAVITIFLLGAGFLASGGFGVAALSVLSWIYRYLTGKHPPGADQLESAKTKLASKAREMKDRAEQFSQQPVAGSQTS

SEQ ID NO: 4 - protein, 138 aa, Pinus massoniana
MQKIHDHTPNPTQILGFITLFVSGAVLLFLTGLTLTGTVVGLVVLTPVLIFFSPILIPLATVLFVAVAGFLSAGGFGLAALSAISWLYNYIKGRHPPGADQIDYARMRIADTATHVKDYAREYGGYLQSKIQDAAPGA

SEQ ID NO: 5 - protein, 149 aa, Brassica napus
MANQTRTHQDIIVRDSRSTLDRDHPKTGAQMVKVATGVAAGGSLLVLSGLTLAGTVIALAVATPLLIIFSPVLVPAVITVVLIITGFLASGGFGIAAITAFSWLYRHMTGSGSDQKIESARMKVGSRGYDTKSGQHNIGVHQQHQQAAS

SEQ ID NO: 6 - protein, 156 aa, Zea mays
MADHHRGATGGGGGYGDLQRGGGMHGEAQQQQKQGAMMTALKAATAATFGGSMLVLSGLILAGTVIALTVATPVLVIFSPVLVPAAIALALMAAGFVTSGGLGVAALSVFSWMYKYLTGKHPPAADQLDHAKARLASKARDVKDAAQHRIDQAQGS

SEQ ID NO: 7 - DNA, 516 bp, Arabidopsis thaliana
atggcggatacagctagaggaacccatcacgatatcatcggcagagaccagtacccgatgatgggccgagaccgagaccagtaccagatgtccggacgaggatctgactactccaagtctaggcagattgctaaagctgcaactgctgtcacagctggtggttccctccttgttctctccagccttacccttgttggaactgtcatagctttgactgttgcaacacctctgctcgttatcttcagcccaatccttgtcccggctctcatcacagttgcactcctcatcaccggttttctttcctctggagggtttggcattgccgctataaccgttttctcttggatttacaagtacgcaacgggagagcacccacagggatcagacaagttggacagtgcaaggatgaagttgggaagcaaagctcaggatctgaaagacagagctcagtactacggacagcaacatactggtggggaacatgaccgtgaccgtactcgtggtggccagcacact

SEQ ID NO: 8 - DNA, 531 bp, Arachis hypogaea
atggctactgctactgatcgtgcacctcaccaggttcaagttcacacccccaccacacaacgcgtcgacgttccacgccgcggctacgatgttagtggtggtggtattaagactcttctccccgagagaggtccgtccacctctcaaatcatcgccgtcctcgtcggcgtccccactgggggcactctgttgctcctctccggcctttcacttctcggaaccataatcgggctggcaattgccaccccggtttttatcttcttcagcccggttatagttcccgcggtcgttaccattggacttgcggtcactggtattctcacggcgggagcatgtggactaaccgggctgatgtctttgtcatggatgattaacttcatccgacaggtacatgggacgacggtgccggatcagctggactcagtgaagcggcgcatggcggacatggcggattacgtggggcagaagacaaaggatgctggccaagagatacagactaaggcccaggatgttaagaggtcatcatcataa

SEQ ID NO: 9 - DNA, 438 bp, Sesamum indicum
atggctgagcattatggtcaacaacagcagaccagggcgcctcacctgcagctgcagccgcgcgcccagcgggtagtgaaggcggccaccgccgtgacagccggcggctcgcttctcgtcctctctggcctcacattagccggaactgttattgcgctcaccatcgccactccgctgcttgtgatctttagccccgttctggtgccggcggtcataaccattttcttgctgggtgcgggttttctggcatccggaggcttcggcgtggcggcgctgagtgtgctgtcgtggatttacagatatctgacagggaaacacccgccgggggcggatcagctggaatcggcaaagacgaagctggcgagcaaggcgcgagagatgaaggatagggcagagcagttctcgcagcagcctgttgcggggtctcaaacttcttga

SEQ ID NO: 10 - DNA, 757 bp, Pinus massoniana
atagggcaagcagtggtatcaacgcagagtacatgggaagttagcctctgcaacgctaaaattaagttcatacagcgagtagtttacaggtttttttttgcttttcagggtttttcagggtttttaagcatcatttgagatggctgatcagttcacgaagtgatgcagaaaattcatgatcatacgcccaatccaactcagatcttgggtttcatcaccctgttcgtttctggtgcagttttgctgtttttgactggtctcacattaactggaacggttgtagggttggtggttctcactcctgttctcattttcttcagtcccatattgataccattagcaactgtgctgtttgtagcagttgcagggttcttgtctgcaggaggctttggactggcagccttatctgccatttcatggctttacaactacattaagggcaggcatccccctggggcagaccagatagactatgcccgcatgcgtattgcagatacagcgacccatgttaaggattatgcccgtgaatatggtgggtatttgcagagcaagattcaggatgctgctcccggagcttaagtaaggtcttggaccgtaataaattcaggatatatgcagtatgtatatgctctcatttagctgctcatctgatttccatggggtgaatcagttgttttgcagtacgtgggggtctgtttattttgtgagttgcatcattttctttatggtgaagttctttttgttgttgttttttttt

SEQ ID NO: 11 - DNA, 631 bp, Brassica napus
ggtgtcggagagatcataacaagaatcaatggcgaatcaaacaagaacccatcaagacataatagtccgagacagtagaagtaccctagacagagaccatccgaagacaggggcgcagatggtgaaggtagcgactggtgtcgcagccggtggatccctcctagtcctctcaggcttaacactcgccggtacagtgatagcactcgctgtagccactcctctgcttattatatttagccctgtcttggtcccagcggtgatcaccgtggttctcatcatcactgggttccttgcctccggtggctttggtatagccgccataaccgccttctcttggctctacaggcacatgacggggtctggatcggatcaaaagatagagagtgctcgaatgaaggtgggaagtagagggtatgataccaagtctgggcagcacaacattggagtccatcagcaacaccaacaagcagcttcttaaaattaatgtcagataaaactatttccacacaaattgtcatatccatcgtttgatctcttttcttcgttttctcactgcttgtgttgtaacgtaacgagtaataataacatgagtgtatgttgcccaaaaaaaaaaaaaaaaaaaaaaaaaaaa

SEQ ID NO: 12 - DNA, 757 bp, Zea mays
cggggtggcgggggctacggcgacctccagcgcgggggcggcatgcacggcgaggcgcagcagcagcagaagcagggcgccatgatgacggcgctcaaggccgcgacggccgcgaccttcggcgggtcgatgctggtgctgtccgggctgatcctggccggcaccgtgatcgcgctcacggtggccacccccgtgctggtgatcttcagcccggtgctggtgccagccgccatcgcgctggcgctcatggcggccgggttcgtcacctccggcggcctcggcgtcgctgcgctgtccgtgttctcctggatgtacaagtacctgacgggcaagcacccgccgggcgccgaccagctggaccacgccaaggcgaggctggcgtccaaggcccgcgacatcaaggatgcagcacagcaccgcatcgaccaggcgcaggggtcttgagagaagaaccacactcgagcggaccgcgcgcgcgtcctccttgaaccactgccggcgcggcggcatatggcccttaaaggcggtggctgctgctacgtacgctgccgtagagtctcggtcgccgcgatagctctagctagtcgtttatgtgttgtgctttgtgtgtgcatgcatgtgtctgggggcatgcagtcagtgcagtactatatgctgtatgcgtctctctttgatcggagaggcggatgtacagcatgctcgatatgtctagtttggatgtcatgtttatgatgaggaataaaatgcaggtcaggtg

SEQ ID NO: 13 - protein, 12 aa, Arabidopsis thaliana
PLFLIFSPVIVP

SEQ ID NO: 14 - protein, 12 aa, Oryza sativa
PVFLIFSPVLVP

SEQ ID NO: 15 - protein, 12 aa, Populus trichocarpa
PLFILFSPVLVP

SEQ ID NO: 16 - protein, 12 aa, Selaginella mellidorfii
PVFLFFSPVLVP

SEQ ID NO: 17 - protein, 12 aa, Psycomitrella patens
PLLIIFSPILIP

SEQ ID NO: 18 - protein, 12 aa, Marchantia polymorpha
PVLVFFSPILIP

SEQ ID NO: 19 - DNA, 36 bp, Marchantia polymorpha
cctgttttggtattcttcagtccaatcttgatacct

SEQ ID NO: 20 - protein, 85 aa, Brassica napus
IAKAVTAVTAGGSLLVLSSLTLVGTVIALTVATPLLVIFSPILVPALITVALLITGFLSSGGFGIAAITVFSWIYKYATGEHPQG

SEQ ID NO: 21 - DNA, 255 bp, Brassica napus
attgctaaggctgttaccgcagtcacggccggtgggtcccttcttgtcctctccagtctcacccttgtcggaactgtcattgctctgactgttgcgactcctctgcttgttatctttagtccaatccttgtccctgctctcatcaccgttgcattgctcatcaccggctttctctcctctggtggctttggcattgcagctataaccgtcttctcttggatctacaagtatgcaacgggagagcacccacaaggg

SEQ ID NO: 22 - protein, 424 aa, Artificial Sequence
  Oleosin- CFP Fusion polypeptide
MADTARGTHHDIIGRDQYPMMGRDRDQYQMSGRGSDYSKSRQIAKAATAVTAGGSLLVLSSLTLVGTVIALTVATPLLVIFSPILVPALITVALLITGFLSSGGFGIAAITVFSWIYKYATGEHPQGSDKLDSARMKLGSKAQDLKDRAQYYGQQHTGGEHDRDRTRGGQHTDYKDDDDKENLYFQGSMVSKGEELVELDGDVNGHKFSVSGEGEGDATYGKLTLKFICTTGKLPVPWPTLVTTLTWGVQCFSRYPDHMKQHDFFKSAMPEGYVQERTIFFKDDGNYKTRAEVKFEGDTLVNRIELKGIDFKEDGNILGHKLEYNYISHNVYITADKQKNGIKANFKIRHNIEDGSVQLADHYQQNTPIGDGPVLLPDNHYLSTQSALSKDPNEKRDHMVLLEFVTAAGITLGMDELGMDELYK

SEQ ID NO: 23 - protein, 45 aa, Arabidopsis thaliana
MADTARGTHHDIIGRDQYPMMGRDRDQYQMSGRGSDYSKSRQIAK

SEQ ID NO: 24 - protein, 72 aa, Arabidopsis thaliana
AATAVTAGGSLLVLSSLTLVGTVIALTVATPLLVIFSPILVPALITVALLITGFLSSGGFGIAAITVFSWIY

SEQ ID NO: 25 - protein, 55 aa, Arabidopsis thaliana
KYATGEHPQGSDKLDSARMKLGSKAQDLKDRAQYYGQQHTGGEHDRDRTRGGQHT

SEQ ID NO: 26 - DNA, 135 bp, Arabidopsis thaliana
atggcggatacagctagaggaacccatcacgatatcatcggcagagaccagtacccgatgatgggccgagaccgagaccagtaccagatgtccggacgaggatctgactactccaagtctaggcagattgctaaa

SEQ ID NO: 27 - DNA, 216 bp, Arabidopsis thaliana
gctgcaactgctgtcacagctggtggttccctccttgttctctccagccttacccttgttggaactgtcatagctttgactgttgcaacacctctgctcgttatcttcagcccaatccttgtcccggctctcatcacagttgcactcctcatcaccggttttctttcctctggagggtttggcattgccgctataaccgttttctcttggatttac

SEQ ID NO: 28 - DNA, 165 bp, Arabidopsis thaliana
aagtacgcaacgggagagcacccacagggatcagacaagttggacagtgcaaggatgaagttgggaagcaaagctcaggatctgaaagacagagctcagtactacggacagcaacatactggtggggaacatgaccgtgaccgtactcgtggtggccagcacact

SEQ ID NO: 29 - protein, 7 aa, Artificial Sequence
  Synthetic Linker
ENLYFQS

SEQ ID NO: 30 - DNA, 21 bp, Artificial Sequence
  Synthetic linker
gagaacctctacttccaatcg

SEQ ID NO: 31 - protein, 198 aa, Artificial Sequence
  Synthetic Linker (intein)
CITGDALVALPEGESVRIADIVPGARPNSDNAIDLKVLDRHGNPVLADRLFHSGEHPVYTVRTVEGLRVTGTANHPLLCLVDVAGVPTLLWKLIDEIKPGDYAVIQRSAFSVDCAGFARGKPEFAPTTYTVGVPGLVRFLEAHHRDPDAQAIADELTDGRFYYAKVASVTDAGVQPVYSLRVDTADHAFITNGFVSHA

SEQ ID NO: 32 - DNA, 594 bp, Artificial Sequence
  Synthetic linker (Intein)
tgcatcacgggagatgcactagttgccctacccgagggcgagtcggtacgcatcgccgacatcgtgccgggtgcgcggcccaacagtgacaacgccatcgacctgaaagtccttgaccggcatggcaatcccgtgctcgccgaccggctgttccactccggcgagcatccggtgtacacggtgcgtacggtcgaaggtctgcgtgtgacgggcaccgcgaaccacccgttgttgtgtttggtcgacgtcgccggggtgccgaccctgctgtggaagctgatcgacgaaatcaagccgggcgattacgcggtgattcaacgcagcgcattcagcgtcgactgtgcaggttttgcccgcgggaaacccgaatttgcgcccacaacctacacagtcggcgtccctggactggtgcgtttcttggaagcacaccaccgagacccggacgcccaagctatcgccgacgagctgaccgacgggcggttctactacgcgaaagtcgccagtgtcaccgacgccggcgtgcagccggtgtatagccttcgtgtcgacacggcagaccacgcgtttatcacgaacgggttcgtcagccacgct

SEQ ID NO: 33 - DNA, 496 bp, Artificial Sequence
  Synthetic - promoter Hsp70A-Rbc52
tcgctgaggcttgacatgattggtgcgtatgtttgtatgaagctacaggactgatttggcgggctatgagggcgggggaagctctggaagggccgcgatggggcgcgcggcgtccagaaggcgccatacggcccgctggcggcacccatccggtataaaagcccgcgaccccgaacggtgacctccactttcagcgacaaacgagcacttatacatacgcgactattctgccgctatacataaccactcagctagcttaagatcccatcaagcttgcatgccgggcgcgccagaaggagcgcagccaaaccaggatgatgtttgatggggtatttgagcacttgcaacccttatccggaagccccctggcccacaaaggctaggcgccaatgcaagcagttcgcatgcagcccctggagcggtgccctcctgataaaccggccagggggcctatgttctttacttttttacaagagaagtcactcaacatcttaaa

SEQ ID NO: 34 - protein, 199 aa, Arabidopsis thaliana
MADTHRVDRTDRHFQFQSPYEGGRGQGQYEGDRGYGGGGYKSMMPESGPSSTQVLSLLIGVPVVGSLLALAGLLLAGSVIGLMVALPLFLLFSPVIVPAALTIGLAMTGFLASGMFGLTGLSSISWVMNYLRGTRRTVPEQLEYAKRRMADAVGYAGQKGKEMGQHVQNKAQDVKQYDISKPHDTTTKGHETQGRTTAA

SEQ ID NO: 35 - protein, 141 aa, Arabidopsis thaliana
MADQTRTHHEMISRDSTQEAHPKARQMVKAATAVTAGGSLLVLSGLTLAGTVIALTVATPLLVIFSPVLVPAVVTVALIITGFLASGGFGIAAITAFSWLYRHMTGSGSDKIENARMKVGSRVQDTKYGQHNIGVQHQQVS

SEQ ID NO: 36 - protein, 191 aa, Arabidopsis thaliana
MANVDRDRRVHVDRTDKRVHQPNYEDDVGFGGYGGYGAGSDYKSRGPSTNQILALIAGVPIGGTLLTLAGLTLAGSVIGLLVSIPLFLLFSPVIVPAALTIGLAVTGILASGLFGLTGLSSVSWVLNYLRGTSDTVPEQLDYAKRRMADAVGYAGMKGKEMGQYVQDKAHEARETEFMTETHEPGKARRGS

SEQ ID NO: 37 - DNA, 925 bp, Arabidopsis thaliana
attacaaagaaaataggtaaaaacaatttctcattagcttacaatggcggatacacaccgtgtcgaccgtactgatagacactttcaatttcagtcgccctatgaaggcggccgaggtcaaggtcagtatgaaggtgaccgtggttacggtggtggcggttacaagagcatgatgcctgaaagtggcccatctagtacccaagtattgtccctgttgattggagtccctgtcgtcggttcgctacttgccttggctggattacttctagctggttcggtgatcggcttaatggttgctttaccactatttctcctcttcagcccggttatagtcccagcggctctaactatcgggcttgcaatgacaggctttttagcctcggggatgttcggtctaaccgggcttagctcaatctcatgggtcatgaactatcttcgtgggacaaggagaactgtgcctgagcaattggagtatgctaagaggagaatggctgatgcggttggctacgcaggacaaaagggcaaagaaatgggccagcatgtgcagaacaaggcccaagatgttaaacaatatgatatttctaagccacatgacactaccactaagggtcatgagactcaggggaggacgacggctgcatgatgagttttcagtatgaacggtagatatgtgttttcactattatgtcgttttttctgcattttcaatatgatgttatgtgttttttttgtttggctttttgttgaaccgtgtatgtgttttatgtttttgtaagcatgaaagatcgcaagtgttgtggtaatatttgaatgtaataatatgataagttgataaatcatgggaacatttaaattaggtggacatgtttagctatttgatactcacagttctttaggtttctagttttttttttgtctatataga

SEQ ID NO: 38 - DNA, 767 bp, Arabidopsis thaliana
ccttcataaatcatcattacttactccgaaaacacatcactaccttttctctgctaatttctactatttgtgttggagggatatattataataaaaagaaccaatggcggaccaaacaagaacccatcacgagatgataagccgagacagtacccaagaggcccatccgaaggccaggcagatggtgaaggcagcaaccgctgtcacagccggtggatccctacttgtcctctccggcttaacactcgctggaacagtcatcgcactcacggtggctactcctctcctcgtcatcttcagccccgtcttggttccagcagtggtaaccgttgctctcatcattaccggattccttgcatccggtggctttggaatagccgccattaccgccttctcttggctctacaggcacatgacgggatctggatcggataagatagagaatgctcggatgaaggttggaagcagagtgcaggatactaagtatgggcagcacaacattggagtccaacaccaacaagtttcttaaaattcaaaggctaaaaacgatttccacattaaattgtcatccatcgttagatctcttctctttgtttttattttcaacgtttatgttgtaatgtgtgatatataataacatgcaatgtatgatgtaatcttgttgttgttgtatgagtaatgttttgatccttctatgcgagcggaaatcttcatatagacagtgtcattggtacattacacaaatcttcataatctccttctacgct

SEQ ID NO: 39 - DNA, 1385 bp, Arabidopsis thaliana
catcatcctacattcatacctaagctagcaaagcaaactactaaaagggtcgtcaacgacaagttatttgctagttggtgcatactacacacggctacggcaacattaagtaacacattaagaggtgttttcttaatgtagtatggtaattatatttatttcgaaacttggattagatataaaggtacagttagtgaaaatatttggttagcgggttgaattaaccggatataggagtcatatatacaactgtgaaaaaaggataaatacaaaaagggaagatgtttttccgacacacaagctaattaagtgcatcgagaggagagcaattgtaaaatgaatgttttgtttgtttttgtacggtggagagaagaacgaaaagatgatcaggtaaaaaatgaaacttggaaatcatgcaaagccacacctctcccttcaacacattcttacgtgtcgtcttctcttcactccatatctcctttttattaccaacaaatatatttcaatcccatttatatgtacgttctcgtagacttatctctatataccccctttaatttgtttgctcttagcctttactttatagttttatatcatatcaatcgacatggcgaatgtggatcgtgatcggcgtgtgcatgtagaccgtactgacaaacgtgttcatcagccaaactacgaagatgatgtcggttttggtggctatggcggttatggtgctggttctgattataagagtcgcggcccctccactaaccaaatcttggcacttatagcaggagttcccattggtggcacactgctaaccctagctggactcactctagccggttcggtgatcggcttgctagtctccatacccctcttcctcctcttcagtccggtgatagtcccggcggctctcactattgggcttgctgtgacgggaatcttggcttctggtttgtttgggttgacgggtctgagctcggtctcgtgggtcctcaactacctccgtgggacgagtgatacagtgccagagcaattggactacgctaaacggcgtatggctgatgcggtaggctatgctggtatgaagggaaaagagatgggtcagtatgtgcaagataaggctcatgaggctcgtgagactgagttcatgactgagacccatgagccgggtaaggccaggagaggctcataagctaatataaattgcgggagtcagttggaaacgcgataaatgtagttttacttttatgtcccagtttctttcctcttttaagaatatctttgtctatatatgtgttcgttcgttttgtcttgtccaaataaaaatccttgttagtgaaataagaaatgaaataaatatgttttcttttttgagataaccagaaatctcatac